(19) United States
(12) Patent Application Publication
(10) Pub. No.: US 2010/0041065 A1
(43) Pub. Date: Feb. 18, 2010

(54) DETECTION METHOD, SAMPLE CELL AND KIT FOR DETECTION AND DETECTION APPARATUS

(75) Inventors: Kazuyoshi Horii, Ashigarakami-gun (JP); Bo Yang, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/539,111

(22) Filed: Aug. 11, 2009

(30) Foreign Application Priority Data
Aug. 12, 2008 (JP) 2008-207934

(51) Int. Cl. G01N 33/53; C12M 1/34

(52) U.S. Cl. 435/7.1; 435/287.2

(57) ABSTRACT

A labeling binding substance in an amount corresponding to the amount of a detection target substance contained in a liquid sample binds to a sensor portion, and the amount of the detection target substance is detected based on the amount of signal light output by excitation of a label of the labeling binding substance in an enhanced optical field on the sensor portion. In this detection method, a labeling substance that includes a light-responsive substance enclosed by a dielectric that transmits light output from the light-responsive substance is used as the label, and the labeling binding substance binds to the sensor portion through a plurality of fragmented antibodies.

### BACKGROUND OF THE INVENTION

1. Field of the Invention

The present invention relates to a detection method, a sample cell for detection, a kit for detection and a detection apparatus that detect a substance to be detected (a detection target substance) in a sample.

2. Description of the Related Art

Conventionally, in the field of bio-measurement and the like, a fluorescence detection method is widely used as a highly accurate and easy measurement method. In the fluorescence detection method, a sample that is presumed to contain a detection target substance that outputs fluorescence by excitation by irradiation with light having a specific wavelength is irradiated with excitation light having the specific wavelength. At this time, fluorescence is detected to confirm presence of the detection target substance. Further, when the detection target substance is not a phosphor (fluorescent substance), a substance that has been labeled with a fluorescent dye and that specifically binds to the detection target substance is placed in contact with the sample. Then, fluorescence from the fluorescent dye is detected in a manner similar to the aforementioned method, thereby confirming presence of a bond between the detection target substance and the substance that specifically binds to the detection target substance. In other words, presence of the detection target substance is confirmed, and this method is widely used.

In bio-measurement, an assay is performed, for example, by using a sandwich method, a competition method or the like. In the sandwich method, when an antigen, as a detection target substance, contained in a sample needs to be detected, a primary antibody that specifically binds to the detection target substance is immobilized on a substrate (base), and a sample is supplied onto the substrate to make the detection target substance specifically bind to the primary antibody. Further, a secondary antibody to which a fluorescent label has been attached, and that specifically binds to the detection target substance, is added to make the secondary antibody bind to the detection target substance. Accordingly, a so-called sandwich structure of (primary antibody)-(detection target substance)-(secondary antibody) is formed, and fluorescence from the fluorescent label attached to the secondary antibody is detected. In the competition method, a competitive secondary antibody that competes with the detection target substance, and that specifically binds to a primary antibody, and to which a fluorescent label has been attached, binds to the primary antibody in such a manner to compete with the detection target substance. Further, fluorescence from the competitive secondary antibody that has bound to the primary antibody is detected.

When the assay is performed as described above, an evanescent fluorescence method has been proposed. In the evanescent fluorescence method, fluorescence is excited by evanescent light to detect fluorescence only from the secondary antibody that has bound, through the detection target substance, to the primary antibody immobilized on the substrate, or fluorescence only from the competitive secondary antibody that has directly bound to the primary antibody. In the evanescent fluorescence method, fluorescence excited by evanescent waves that extend from the surface of the substrate is detected. The evanescent waves are generated by making excitation light that totally reflects on the surface of the substrate enter the substrate from the back side of the substrate.

In the evanescent fluorescent method, methods using electric-field enhancement effects by plasmon resonance are proposed to improve the sensitivity of detection in U.S. Pat. No. 6,194,223 (Patent Literature 1), “Surface Plasmon Fluorescence Measurements of Human Chorionic Gonadotrophin: Role of Antibody Orientation in Obtaining Enhanced Sensitivity and Limit of Detection”, M. M. L. M. Vareiro et al., Analytical Chemistry, Vol. 77, pp. 2426-2431, 2005 (Non-Patent Literature 1), and the like. In a surface plasmon enhancement fluorescence method, a metal layer is provided on the substrate, and excitation light is caused to enter the interface between the substrate and the metal layer from the back side of the substrate at an angle greater than or equal to a total reflection angle to generate surface plasmon resonance in the metal layer. Further, fluorescent signals are enhanced by the electric field enhancement effect of the surface plasmons to improve the S/N (signal to noise) ratio.

Similarly, in the evanescent fluorescence method, a method using electric field enhancement effects by a waveguide mode is proposed in “High-sensitive sensing of catechol amines using by optical waveguide mode enhanced fluorescence spectroscopy”, K. Tsuboi et al., the Japan Society of Applied Physics, Collection of Presentation Abstracts, No. 3, p. 1378, 2007 (Non-Patent Literature 2). In this optical waveguide mode enhanced fluorescence spectroscopy (OWF), a metal layer and an optical waveguide layer including a dielectric and the like are sequentially formed on the substrate. Further, excitation light is caused to enter the substrate from the back side of the substrate at an angle that is greater than or equal to the total reflection angle to induce an optical waveguide mode in the optical waveguide layer by irradiation with the excitation light. Further, fluorescent signals are enhanced by the electric field enhancement effect by the optical waveguide mode.

Further, Specification of U.S. Patent Application Publication No. 20050053974 (Patent Literature 2) and “Surface-plasmon field-enhanced fluorescence spectroscopy”, T. Liebermann and W. Knoll, Colloids and Surfaces A, Vol. 171, pp. 115-130, 2000 (Non-Patent Literature 3) propose a method for extracting radiation light (SPCE: Surface Plasmon-Coupled Emission) from the prism side. In the method, instead of detecting fluorescence output from a fluorescent label excited in the electric field enhanced by surface plasmons, the fluorescence newly induces surface plasmons in the metal layer, and radiation light by the newly induced plasmons is extracted from the prism side.

It is well known that the electric field enhanced by using the evanescent fluorescent method sharply attenuates as a distance from the enhanced-electric-field generation surface increases. FIG. 20 is a graph showing a result of simulation by the inventors of the present invention. In the simulation, the dependency characteristic of the electric field enhancement effect on a distance from the enhanced electric field generation surface (metal surface) was simulated for a case in which a laser beam having a wavelength of 656 nm entered at an incident angle of 72.5° in a prism (PMMA) —gold layer (gold film or thin-film) (thickness of the gold layer is 50 nm) —solvent (water) system. As confirmed in FIG. 20, in surface plasmon fluorescence detection (SPF) and SPCE, when the distance from the enhanced electric field generation surface is approximately 100 nm, the intensity (magnitude or strength) of the electric field becomes a half of the intensity of the electric field at the enhanced electric field generation surface. Therefore, it is desirable that the fluorescent label is located as close to the enhanced electric field generation surface as possible.

Meanwhile, when the metal layer is exposed to the sample contact surface in the evanescent fluorescent method, if the fluorescent dye in the sample and the metal layer are too close to each other, energy excited in the fluorescent dye is transferred to the metal layer before fluorescence is generated by energy excited in the fluorescent dye, and fluorescence is not generated (so-called metal quenching occurs). Therefore, when the metal layer is exposed to the sample contact surface, it is necessary to make the fluorescent dye and the metal layer be apart from each other by maintaining a sufficient distance between the fluorescent dye and the enhanced electric field generation surface to prevent metal quenching or by providing a metal oxide layer or a dielectric layer, such as a polymer layer, a three-dimensional layer, such as carboxy methyl dextran (CMD), or the like on the metal layer (“Surface Plasmon Fluorescence Immunoassay of Free Prostate-Specific Antigen in Human Plasma at the Femtomolar Level”, F. Yu et al., Analytical Chemistry, Vol. 76 pp. 6765-6770, 2004 (Non-Patent Literature 4), or the like).

As described above, the effective use of the enhanced electric field and prevention of metal quenching contradict each other. Therefore, a detection method that can effectively prevent metal quenching and efficiently use the enhanced electric field at the same time is needed to achieve highly sensitive detection. This problem is not limited to the fluorescent detection method but common to detection methods using light-responsive substance as a label.

### SUMMARY OF THE INVENTION

In view of the foregoing circumstances, it is an object of the present invention to provide a detection method and apparatus for directly or indirectly detecting optical signals, such as fluorescence and scattered light, at high sensitivity by preventing metal quenching and by efficiently amplifying the intensity of signals at the same time.

Further, it is another object of the present invention to provide a sample cell and a kit for detection that are used in the detection method of the present invention.

A detection method of the present invention is a detection method comprising the steps of:

preparing a sensor chip including a dielectric plate and a sensor portion that has a metal layer deposited on a surface of the dielectric plate;

binding a labeling binding substance in an amount corresponding to the amount of a detection target substance contained in a liquid sample to the sensor portion by contacting the liquid sample with the sensor portion;

irradiating the sensor portion with excitation light to generate an enhanced optical field on the sensor portion; and

detecting the amount of the detection target substance based on the amount of light generated from a label of the labeling binding substance in the enhanced optical field, wherein a fluorescent substance including a light-responsive substance enclosed by a dielectric that transmits light output from the light-responsive substance is used as the label, and wherein the labeling binding substance binds to the sensor portion through a plurality of fragmented antibodies.

Here, the “labeling binding substance” is a binding substance to which a label has been attached. The binding substance in an amount corresponding to the amount of the detection target substance binds to the surface of the sensor portion. For example, when an assay by a sandwich method is performed, the labeling binding substance contains a label and a binding substance that specifically binds to the detection target substance. When an assay by a competition method is performed, the labeling binding substance contains a label and a binding substance that competes with the detection target substance.

In the specification of the present application, the “light-responsive substance” means a substance that generates light by irradiation with excitation light. Examples of the light-responsive substance are organic fluorescent dye, inorganic fluorescent dye, such as quantum dots, and metal micro-particles and the like. Further, light output from the light-responsive substance is fluorescence, phosphorescence, scattered light or the like.

Further, the expression “detecting the amount of the detection target substance” means detecting presence of the detection target substance as well as detecting the amount of the detection target substance. Further, the amount of the detection target substance may mean not only the quantitative amount of the detection target substance but the qualitative value of the detection target substance.

Here, the term “optical field” refers to an electric field generated by evanescent light excited by irradiation with excitation light or by near field light.

Further, the expression “generate an enhanced optical field” means that an enhanced optical field is formed by enhancing the optical field. The optical field may be enhanced by plasmon resonance or excitation of optical waveguide mode.

Further, in the method for “detecting the amount of the detection target substance based on the amount of light generated by excitation of a label of the labeling binding substance”, light from the label may be detected directly. Alternatively, the light may be detected indirectly.

Specifically, the amount of the detection target substance may be detected, for example, in any of the following manners (1) to (4):

(1) Plasmons are excited in a metal layer by irradiation with excitation light, and an enhanced optical field is generated by the plasmons. Further, the amount of the detection target substance is detected by detecting, as light generated by excitation of the label, light output from the label by excitation of the label;

(2) Plasmons are excited in the metal layer by irradiation with the excitation light, and an enhanced optical field is generated by the plasmons. Further, the amount of the detection target substance is detected by detecting, as light generated by excitation of the label, radiation light that radiates from the opposite surface of the dielectric plate. The radiation light radiates by newly inducing plasmons in the metal layer by light output from the label by excitation of the label;

(3) The sensor chip includes an optical waveguide layer deposited on the metal layer. An optical waveguide mode is excited in the optical waveguide layer by irradiation with the excitation light, and an enhanced optical field is generated by the optical waveguide mode. Further, the amount of the detection target substance is detected by detecting, as the light generated by excitation of the label, light output from the label by excitation of the label; and

(4) The sensor chip includes an optical waveguide layer deposited on the metal layer. An optical waveguide mode is excited in the optical waveguide layer by irradiation with the excitation light, and an enhanced optical field is generated by the optical waveguide mode. Further, the amount of the detection target substance is detected by detecting, as the light generated by excitation of the label, radiation light that radiates from the opposite-surface of the dielectric plate. The radiation light radiates by newly inducing plasmons in the metal layer by light output from the label by excitation of the label.

In the methods (1) and (2), the metal layer may be a metal film (coating, thin-film or the like). Further, p-polarized excitation light may be caused to enter the interface between the metal film and the substrate from the back side of the substrate at an angle greater than or equal to the total reflection angle to excite surface plasmons on the surface of the metal film. Alternatively, the metal layer may be formed by a metal fine structure body having an uneven pattern on the surface thereof, and the uneven pattern may include projections and depressions at cycles smaller than the wavelength of the excitation light. Alternatively, the metal layer may include a plurality of metal nanorods smaller than the wavelength of the excitation light. The metal layer may be formed in such a manner that localized plasmons are excited in the metal fine structure body or the metal nanorods by irradiation with excitation light.

In the specification of the present application, the fragmented antibody is a part of an antibody molecule. The fragmented antibody has at least one antigenic determinant group (epitope) that specifically binds to the detection target substance. The fragmented antibody has been fragmented by a protease (proteolytic enzyme), and a disulfide bond and/or a thiol group has been exposed by the fragmentation. It is desirable that the fragmented antibody has an antigenic determinant group that can specifically bind to the detection target substance at least at one of the ends of the fragmented antibody. The fragmented antibody is at least one kind of antibody fragment selected from the group consisting of a Fab fragment, F(ab′)₂ fragment, and a Fab′ fragment.

Further, when at least a part of the metal layer is exposed to a sample-contact surface of the sensor portion, and an antibody having a disulfide bond or thiol group exposed to the metal atom of the exposed metal layer, in other words, an antibody having an exposed S atom, is used as the fragmented antibody, the labeling binding substance can bind onto the sensor portion through the fragmented antibody that has directly bound to the sensor portion.

Further, it is desirable to use the labeling binding substance including the label to the surface of which a fragmented antibody that can specifically bind to the detection target substance and/or the plurality of fragmented antibodies has bound.

A second detection method of the present invention is a detection method comprising the steps of:

preparing a sensor chip including a dielectric plate and a sensor portion that has a metal layer deposited on a surface of the dielectric plate and an optical waveguide layer deposited on the metal layer;

binding a labeling binding substance in an amount corresponding to the amount of a detection target substance contained in a liquid sample to the sensor portion by contacting the liquid sample with the sensor portion;

irradiating the sensor portion with excitation light to excite an optical waveguide mode in the optical waveguide layer to generate an enhanced optical field on the sensor portion by the optical waveguide mode; and

detecting the amount of the detection target substance based on the amount of light generated by excitation of a label of the labeling binding substance by exciting the label in the enhanced optical field, wherein the labeling binding substance binds to the sensor portion through a fragmented antibody.

A detection apparatus of the present invention is used in the detection method of the present invention. The detection apparatus is a detection apparatus comprising:

a sensor chip including a dielectric plate and a sensor portion that has a metal layer deposited on a surface of the dielectric plate, the plurality of fragmented antibodies having bound onto the sensor portion;

an excitation-light irradiation optical system that irradiates the sensor portion with excitation light; and

a light detection means that detects light generated from the label of the labeling binding substance in the enhanced optical field generated on the sensor portion by irradiation with the excitation light.

A sample cell for detection of the present invention is used in the detection method of the present invention. The sample cell for detection is a sample cell comprising:

a base that has a flow path in which a liquid sample flows down;

an injection opening for injecting the liquid sample into the flow path, the injection opening being provided on the upstream side of the flow path;

an air hole for causing the liquid sample that has been injected from the injection opening to flow down toward the downstream side of the flow path, the air hole being provided on the downstream side of the flow path; and

a sensor chip portion provided between the injection opening and the air hole in the flow path, wherein the sensor chip portion includes a dielectric plate that is provided at least as a part of the inner wall of the flow path and a sensor portion that has at least a metal layer deposited on a sample-contact surface of the dielectric plate, wherein the plurality of fragmented antibodies have bound to a surface of the sensor portion opposite to the dielectric-plate-side surface of the sensor portion.

Further, an optical waveguide layer may be provided on the metal layer in the sensor portion.

Further, it is desirable that the sample cell for detection of the present invention includes the labeling binding substance immobilized in the flow path on the upstream side of the sensor portion. It is desirable that the labeling binding substance includes, as the label, a labeling substance that contains a light-responsive substance enclosed by a dielectric that transmits light output from the light-responsive substance.

In the sample cell of the present invention, when the labeling binding substance includes a second binding substance that specifically binds to the detection target substance and binds to the fragmented antibody through the detection target substance, the sample cell is suitable for an assay by a so-called sandwich method.

In the sample cell of the present invention, when the labeling binding substance includes a third binding substance that specifically binds to the fragmented antibody, competing with the detection target substance, the sample cell is suitable for an assay by a so-called competition method.

A kit for detection of the present invention is a kit for detection comprising:

the sample cell for detection of the present invention; and

a solution for labeling, wherein the solution for labeling contains the labeling binding substance that includes, as the label, a labeling substance that includes a light-responsive substance enclosed by a material that transmits light output from the light-responsive substance, and wherein the solution for labeling is injected into the flow path to flow down the flow path together with the liquid sample or after the liquid sample has flowed down the flow path.

The present invention has focused on the distance that is necessary to prevent metal quenching and to introduce antibodies to capture antigens, as a factor that restricts the detection sensitivity and the detection limit. The detection sensitivity and the detection limit is restricted, because the distance prevents efficient use of the enhanced electric field in an assay, such as an electric field enhanced fluorescent spectrum method using near field light or the like. Further, the present invention has succeeded in locating the light-responsive substance remarkably close to the electric field enhancement surface, compared with conventional structure, by using a substance that has a metal-quenching-prevention function (anti-metal-quenching function) and that encloses a light-responsive substance and by using a fragmented antibody.

Non-Patent Literature 1 describes that in a sandwich assay by using a surface plasmon electric field enhancement fluorescent spectrum method, use of a Fab antibody as an antibody for capturing an antigen onto the sensing surface makes it possible to control the orientation of the antigenic determinant group (epitope, antigen recognition site). Therefore, it is possible to increase the amount of the antigen (human chorionic gonadotropin (hCG)) that can be captured and the amount of the labeling antibody. Hence, the detection limit is improved.

In the above literature, the orientation of the antibody is controlled by eliminating three-dimensional obstacles by using a straight-chain fragmented antibody Fab. The orientation is controlled to suppress reduction in the antigen that can be captured and the labeling antibody, the reduction being caused by the variation in the immobilization site due to three-dimensional obstacles of ordinary antibodies, which are Y-shaped. Therefore, use of the fragmented antibody as an antibody in the surface plasmon enhanced fluorescent spectrum method is known. However, in the embodiment of the above literature, when the distance from the electric field enhanced surface is too short, the signal attenuates because of metal quenching. Therefore, in the above literature, an idea of locating the fluorescent substance even closer to the electric field enhanced surface does not exist.

Further, U.S. Pat. No. 6,514,770 describes use of a polystyrene latex particle onto which F(ab′)₂, as an anti-AFP antibody, has been immobilized, as a latex immunoreagent. However, U.S. Pat. No. 6,514,770 fails to even remotely describe that use of F(ab′)₂ reduces the distance between the particle and the antigen.

Further, use of a fragmented antibody alone is insufficient to solve the problem to be solved by the present invention as described above. In the present invention, in addition to the use of the fragmented antibody, a labeling substance having a metal-quenching prevention function is used to solve the problem. Hence, it would not be easy to conceive of the present invention based on the above literature.

In the detection method of the present invention, a labeling substance that includes a light-responsive substance enclosed by a dielectric that transmits light output from the light-responsive substance is used as the label of the labeling binding substance that binds onto the sensor portion based on the amount of the detection target contained in the sample. This labeling substance includes the light-responsive substance enclosed by the dielectric. Therefore, the amount of the light-responsive material that can be used for sensing is large. Hence, it is possible to obtain a stronger optical signal. Further, since the labeling substance per se has a metal-quenching prevention function, it is possible to locate the labeling substance as close to the enhanced electric field generation surface as possible.

Further, in the detection method of the present invention, the labeling binding substance binds onto the sensor portion through the fragmented antibody. Therefore, the distance between the label and the enhanced electric field generation surface is shorter, compared with a case of binding the labeling binding substance through an ordinary antibody. Further, the fragmented antibody has a small number of three-dimensional obstacles. When the antibody has a disulfide bond and/or a thiol group that has been exposed by fragmentation, the antigenic determinant group tends to be oriented to the sample side. Therefore, the density of immobilized antibodies is high, and the rate of non-specific adsorption is low.

Therefore, the present invention can effectively prevent metal quenching and efficiently use the enhanced electric field to directly or indirectly detect an optical signal at high sensitivity.

Further, if the sample cell for detection of the present invention or the kit for detection of the present invention is used, it is possible to easily carry out the detection method of the present invention. Further, it is possible to effectively use the enhanced optical field, and detect presence and/or the amount of the detection target substance at high sensitivity.

### BRIEF DESCRIPTION OF THE DRAWINGS

FIG. 1 is a schematic diagram illustrating the structure of a fluorescence detection apparatus according to a first embodiment of the present invention;

FIG. 2 is an enlarged view of a sensor portion of the fluorescence detection apparatus illustrated in FIG. 1;

FIG. 3 is a schematic diagram illustrating an example of a method for preparing a fragmented antibody;

FIG. 4 is a diagram illustrating a design modification example of an excitation light irradiation optical system;

FIG. 5 is a schematic diagram illustrating the structure of a fluorescence detection apparatus according to a second embodiment of the present invention;

FIG. 6A is a schematic diagram illustrating the structure of a sensor portion of a sensor chip used in a fluorescence detection apparatus according to the second embodiment of the present invention;

FIG. 6B is a schematic diagram illustrating the structure of a sensor portion of a sensor chip used in the fluorescence detection apparatus according to the second embodiment of the present invention;

FIG. 6C is a schematic diagram illustrating the structure of a sensor portion of a sensor chip used in the fluorescence detection apparatus according to the second embodiment of the present invention;

FIG. 7 is a schematic diagram illustrating the structure of a detection apparatus according to a third embodiment of the present invention;

FIG. 8 is a schematic diagram illustrating the structure of a detection apparatus according to a fourth embodiment of the present invention;

FIG. 9 is a schematic diagram illustrating the structure of a detection apparatus according to a fifth embodiment of the present invention;

FIG. 10 is a schematic diagram illustrating a fluorescent substance having a metal coating;

FIG. 11A is a plan view of a sample cell according to the first embodiment of the present invention;

FIG. 11B is a sectional side view of the sample cell according to the first embodiment of the present invention;

FIG. 12 is a diagram illustrating the procedure of an assay by a sandwich method using the sample cell according to the first embodiment of the present invention;

FIG. 13 is a sectional side view illustrating the sample cell according to the second embodiment of the present invention;

FIG. 14 is a diagram illustrating the procedure of an assay by a competition method using the sample cell according to the second embodiment of the present invention;

FIG. 15 is a sectional side view illustrating a design modification example of a sample cell;

FIG. 16 is a schematic diagram illustrating the structure of a kit for detecting fluorescence according to an embodiment of the present invention;

FIG. 17 is a diagram illustrating the procedure of an assay by a sandwich method using the kit for detecting fluorescence;

FIG. 18 is a graph showing the ratio of the fluorescent signal amounts of Examples and Comparative Examples;

FIG. 19 is a graph showing the ratios of the signal amounts of Examples and Comparative Example 1 relative to Comparative Example 2; and

FIG. 20 is a graph showing a dependency characteristic of an electric field enhancement effect by surface plasmons on a distance from an enhanced electric field generation surface.

### DESCRIPTION OF THE PREFERRED EMBODIMENTS

Hereinafter, embodiments of the present invention will be described with reference to the drawings. In each of the diagrams, the size of each unit or element differs from the actual size thereof for the purpose of explanation.

### “Detection Method and Apparatus”

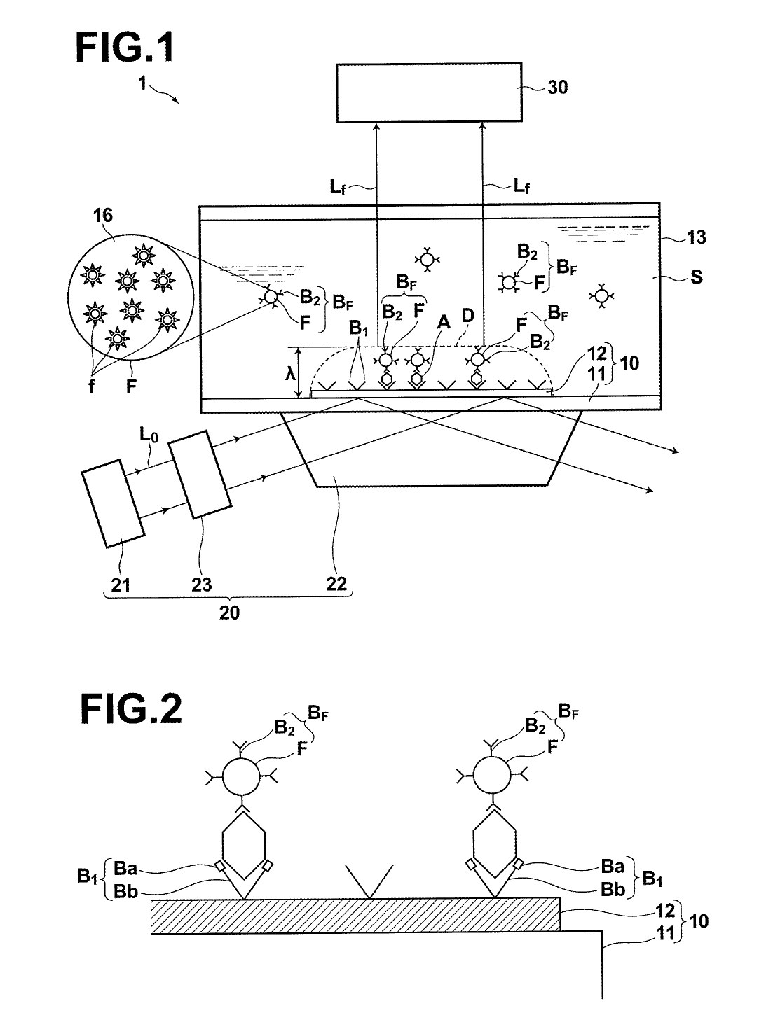

A detection method of the present invention is a light detection method utilizing an enhanced optical field using evanescent light or near field light. For example, a sensor chip 10 including a dielectric plate 11 and a sensor portion 14, as illustrated in FIG. 1, is used. The sensor portion 14 includes at least a metal layer 12 deposited on a surface of the dielectric plate 11. Further, liquid sample S is placed in contact with the sensor portion 14 to make labeling binding substance (fluorescent-label binding substance) B_{F} in an amount corresponding to the amount of detection target substance A (substance to be detected) contained in the liquid sample S bind onto the sensor portion 14. Further, the sensor portion 14 is irradiated with excitation light L₀ to generate an enhanced optical field D on the sensor portion 14, and a label (fluorescent label) F in the labeling binding substance B_{F} is excited in the enhanced optical field D. Further, the amount of the detection target substance A is detected based on the amount of signal light L_{f} generated by excitation of the label F. In the detection method of the present invention, a labeling substance (fluorescent substance) F including a light-responsive substance (fluorescent dye molecules) f enclosed by a material that transmits light output from the light-responsive substance f is used as the label F. Further, the labeling binding substance B_{F} binds to the sensor portion 14 through a plurality of fragmented antibodies B₁.

It is desirable that after the labeling binding substance B_{F} has bound to the sensor portion 14, resident (remaining) or non-specifically-adsorbed labeling binding substance B_{F}, which has not bound to the sensor portion 14, in the liquid sample S is removed from the sensor portion 14, and after then, the amount of the detection target substance A is detected.

Detection apparatuses 1 through 5 according embodiments of the present invention, which will be described below, are used to carry out the aforementioned detection method. Each of the detection apparatuses 1 through 5 includes a housing unit 13, an excitation light irradiation optical system 20, and a light detection means 30. The housing unit 13 houses the sensor chip 10, and the excitation light irradiation optical system 20 includes an excitation light source 21, and irradiates the sensor portion 14 with excitation light L₀. The light detection means 30 detects the amount of signal light L_{f} generated by excitation of the label f in the enhanced optical field D that has been generated on the sensor portion 14 by irradiation with the excitation light L₀.

In the detection method of the present invention, the fragmented antibody B₁ that is provided between the sensor portion 14 of the sensor chip 10 and the labeling binding substance B_{F} may be immobilized on the sensor portion 14 of the sensor chip 10 in advance. Alternatively, the fragmented antibody B₁ may be immobilized on the sensor portion 14 during light detection. In an embodiment of the fluorescence detection apparatus that will be described below, a case in which the fragmented antibody B₁ has been immobilized on the sensor portion 14 of the sensor chip 10 in advance is used as an example.

In the detection method of the present invention, an enhanced optical field is generated on the sensor portion by irradiation with excitation light, and light generated by excitation of a label in the enhanced optical field is detected. The optical field may be enhanced either by surface plasmon resonance or by localized plasmon resonance. Alternatively, the optical field may be enhanced by excitation of optical waveguide mode. Further, the optical signal output from the label may be detected directly or indirectly. Specific examples will be described in each of the embodiments. In the following description of the embodiments, a case in which a fluorescent label is used as the label, and a fluorescent labeling substance that includes a plurality of fluorescent dye molecules enclosed by a dielectric that transmits fluorescence output from the plurality of fluorescent dye molecules is used. Further, the amount of the detection target substance is detected based on the amount of light generated by excitation of the fluorescent label. However, the label and the light that is detected are not limited to these. For example, when a metal micro-particle is used as the light-responsive substance, the amount of the detection target substance should be detected based on the amount of scattered light of excitation light, which is scattered from the micro-particle. Further, the signal amount of the optical signal is increased by the enhanced electric field, regardless of the kind of the optical signal. Further, the problem of metal quenching exists, regardless of the kind of the optical signal. Therefore, the detection method of the present invention can be adopted regardless of the kind of the light-responsive substance and the kind of the optical signal.

### First Embodiment

With reference to FIG. 1, a detection method and apparatus according to a first embodiment of the present invention will be described. FIG. 1 is a schematic diagram illustrating the structure of the whole detection apparatus of the first embodiment. The detection method and apparatus of this embodiment is a fluorescence detection method and apparatus that enhances an optical field by surface plasmon resonance, and detects fluorescence excited in the enhanced optical field.

In the fluorescence detection method of the present embodiment, a sensor chip 10 including a dielectric plate 11 and a sensor portion 14 that has at least a metal film (thin-film, coating, layer or the like), as a metal layer 12, deposited in a predetermined area on a surface of the dielectric plate 11 is used. Further, a plurality of fragmented antibodies B₁ are immobilized on the sensor portion 14 of the sensor chip 10. Further, a sample retaining unit (housing unit) 13 for retaining liquid sample S on the sensor chip 10 is provided. The sensor chip 10 and the housing unit 13, which can retain the liquid sample S, constitute a box-form sample cell.

A fluorescence detection apparatus 1 illustrated in FIG. 1 includes the housing unit 13, the excitation light irradiation optical system 20, and the light detection means 30. The housing unit 13 houses the sensor chip 10. The excitation light irradiation optical system 20 makes linearly polarized excitation light L₀ enter the interface between the dielectric plate 11 and the metal layer 12 of the sensor chip 10, housed in the housing unit 13, at an incident angle that is greater than or equal to a total reflection angle. The linearly polarized excitation light L₀ enters the interface from a surface of the sensor chip 10, the surface being opposite to the metal layer formation surface of the sensor chip 10. The light detection means 30 detects signal light L_{f} that is generated by excitation of the fluorescent label F in the enhanced optical field D that has been generated on the sensor portion 14 by irradiation with the excitation light L₀.

The material of the dielectric plate 11 is not particularly limited as long as the material has a light transmissive characteristic with respect to excitation light L₀. It is desirable that the material of the dielectric plate 11 is an inorganic oxide thin-film, such as SiO₂, TiO₂, and HfO₂, an organic polymer, such as polystyrene, PMMA (polymethyl methacrylate), or the like. In the specification of the present application, the phrase “have a light transmissive characteristic” is defined as transmitting light having a predetermined wavelength.

The metal layer (film, thin-film, coating or the like) 12 may be formed on a surface of the dielectric plate 11 by forming (placing) a mask that has an opening in the predetermined area, and by depositing metal by using a well-known vapor-deposition method (evaporation method). It is desirable that the thickness of the metal layer 12 is appropriately determined, based on the material of the metal layer 12 and the wavelength of the excitation light, so that surface plasmons are strongly excited. For example, when a laser beam that has a center wavelength of 780 nm is used as the excitation light, and a gold (Au) film is used as the metal layer 12, it is desirable that the thickness of the metal layer is 50 nm±20 nm. Further, it is more desirable that the thickness is 47 nm±10 nm. Further, it is desirable that the metal layer contains, as a main component, at least one kind of metal selected from the group consisting of Au, Ag, Cu, Al, Pt, Ni, Ti, and alloys thereof. Here, the term “main component” is defined as a component contained at greater than or equal to 90% by mass.

FIG. 2 is an enlarged view of the surface of the sensor portion 14. As illustrated in FIG. 2, in the sensor portion 14, a plurality of fragmented antibodies B₁ are immobilized on the metal layer 12. As described above, the fragmented antibody B₁ is a part of an antibody molecule, and has at least one antigenic determinant group Bₐ that can bind to the detection target substance A (in FIG. 2, B_{b} is a polypeptide chain other than Bₐ). FIG. 2 shows an example of detecting the amount of the detection target substance A by labeling with a fluorescent-label binding substance B_{F} by using a sandwich method.

Examples of the fragmented antibody B₁ are an antiserum prepared from a serum of an animal immunized by an assay target (analysis target), an immunoglobulin fraction produced from an antiserum, and a fragment of a monoclonal antibody (Fab fragment F(ab′)₂ fragment, Fab′ fragment, Fv, or the like) that is obtained by cell fusion using spleen cells of an animal immunized by the assay target.

The basic structure of an antibody is a Y-shaped four-chain structure, which includes two heavy chains and two light chains. The heavy chains and the light chains bind to each other through disulfide bonds, and form heterodimers. Further, the heterodimers bind to each other by a hinge portion including two disulfide bonds to form a Y-shaped heterotetramer. The upper half of the Y-shape, which is V-shaped, is called as a Fab region (Fab fragment), and the lower half of the Y-shape, which is a straight portion, is called as an Fc region (Fc fragment).

These fragmented antibodies may be prepared by using ordinary methods. For example, an antibody may be fragmented by using a protease (proteolytic enzyme). It is desirable that the antibody is fragmented by using a protease, and a disulfide bond and/or a thiol group is exposed by the fragmentation. Further, it is desirable that the fragmented antibody has, at least at one end thereof, an antigenic determinant group Bₐ that can bind to the fluorescent-label binding substance. Such a fragmented antibody is, for example, at least one kind of antibody fragment selected from the group consisting of a Fab fragment (I-shaped), a F(ab′)₂ fragment (V-shaped), a Fab′ fragment, and a Fv fragment, or the like. Since the antibody that has an exposed disulfide bond and/or an exposed thiol group can directly bind to the metal layer, such as a gold layer, without providing a SAM (self-assembled monolayer) or the like therebetween. Therefore, it is possible to further reduce the distance between the electric field enhancement surface and the fluorescent-label binding substance, and that is desirable. In FIGS. 1 and 2 and other diagrams used in the second embodiment or later embodiments, a V-shaped F(ab′)₂ fragment is used as an example. However, the kind of the fragment is not limited to F(ab′)₂ fragment.

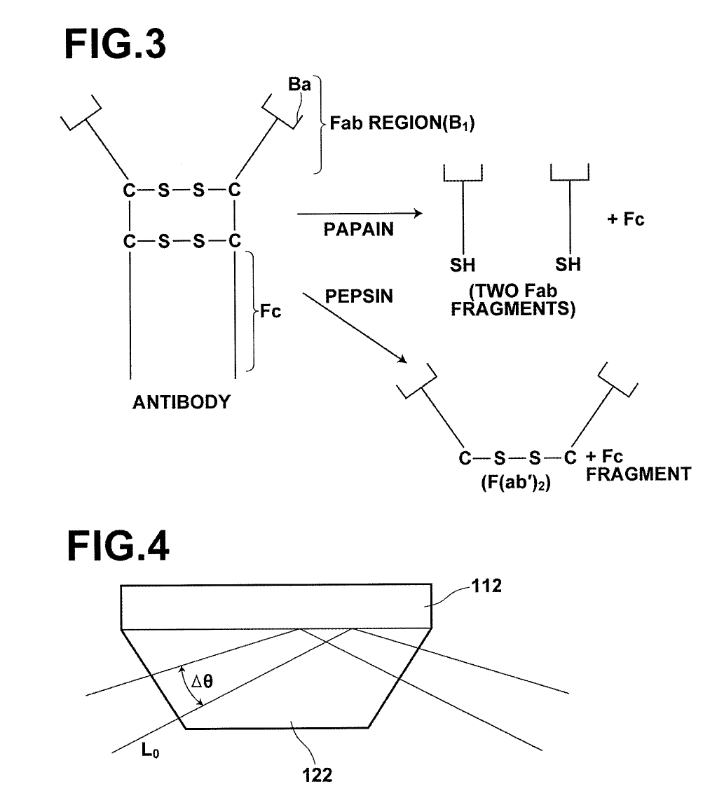

FIG. 3 is a schematic diagram illustrating an example of preparing a fragmented antibody. In FIG. 3, chains of each Fab region are illustrated using a single line to simplify the drawing. As illustrated in FIG. 3, when an antibody is processed with papain, which is a protease, the antibody is broken down into two Fab fragments and an Fc fragment. When an antibody is processed with pepsin, which is a protease, the antibody is broken down into a F(ab′)₂ fragment including two Fab fragments that have bound to each other by a disulfide bond and a fragmented Fc fragment. Further, when the F (ab′)₂ fragment is further processed with a reductant (reducing agent), such as 2-mercaptoethylamine, the F(ab′)₂ fragment is broken into Fab fragments.

Besides the aforementioned enzymes, examples of the enzyme that can produce antibody fragments are ficin, lysyl endopeptidase, V8 protease, bromelain, clostripain, metallo endopeptidase, activated papain, obtained by performing activation processing on papain, or the like.

Further, the fragmentation method is not limited to the aforementioned method using an enzyme. Fragmentation processing may be performed by chemical processing or by using a genetic engineering technique.

A hydrophobic portion at an end of each of the Fc regions of a Y-shaped full antibody molecule more easily binds to the metal layer, compared with the other ends of the antibody. However, the Y-shape often falls down by a three-dimensional obstacle of the Y-shape or the like, and binds to the metal layer in a lying or inclined state. Further, there are often cases in which the antigenic determinant group binds to the surface of the metal layer, which is opposite to an intended binding direction. In contrast, fragmented antibodies have a small number of three-dimensional obstacles, compared with Y-shaped full antibody molecules. Further, since a disulfide bond and/or a thiol group has been exposed by fragmentation, the exposed S atom of the antibody easily binds to metal. Therefore, the antigenic determinant group tends to be oriented to the sample side. Hence, the density of immobilized antibodies becomes high, and non-specific adsorption is reduced.

The fluorescent-label binding substance B_{F} is a binding substance that binds to the sensor portion 14. The binding substance is fluorescence-labeled, and the binding substance in an amount corresponding to the amount of the detection target substance A binds to the sensor portion 14. As illustrated in FIGS. 1 and 2, when an assay by using a sandwich method is performed, the fluorescent-label binding substance B_{F} includes the aforementioned fluorescent substance F and a binding substance B₂ that specifically binds to the detection target substance A. When an assay by using a competition method, which will be described alter, is performed, the fluorescent-label binding substance B_{F} includes the fluorescent substance F and a binding substance that competes with the detection target substance A. Specifically, when the sensor chip 10 that has a sensor portion 14 in which first binding substance B₁ (fragmented antibody) that specifically binds to the detection target substance A is immobilized as an immobilization layer is used, the fluorescent-label binding substance B_{F} is selected in the following manner. When the sandwich method is used, the fluorescent-label binding substance B_{F} including the second binding substance B₂ that specifically binds to the detection target substance A and the fluorescent substance F modified with the binding substance B₂ is used. When the competition method is used, the fluorescent-label binding substance B_{F} including the third binding substance that competes with the detection target substance A and specifically binds to the first binding substance and the fluorescent substance F modified with the third binding substance is used. When the detection target substance A is an antigen, a so-called primary antibody should be used as the first binding substance B₁, and a so-called secondary antibody should be used as the fluorescent-label binding substance.

The second binding substance B₂ may be an ordinary full antibody molecule. However, when a fragmented antibody is used in a manner similar to the aforementioned primary antibody, the distance of bond becomes even shorter, and that is desirable.

As described above, the fluorescent label F is a fluorescent substance including a plurality of fluorescent dye molecules f enclosed by a dielectric 16 that transmits fluorescence L_{f} output from the plurality of fluorescent dye molecules f by excitation by evanescent light (optical field D).

The size of the particle of the fluorescent label F is not particularly limited. However, when a fluorescence amount, highest-density loading of the substance onto the sensor portion, and surface plasmon disturbance are considered, it is desirable that the size of the particle is in the range of 70 nm to 900 nm. Further, it is more desirable that the size of the particle is in the range of 130 nm to 500 nm. In the specification of the present application, when the particle of the fluorescent label F has substantially spherical form, the size of the particle of the fluorescent substance is the diameter of the particle. When the particle does not have spherical form, an average length of the maximum width and the minimum width of the particle is defined as the size of the particle.

Further, the dielectric 16 is not particularly limited as long as the dielectric 16 can enclose the fluorescent dye molecules f and transmit evanescent light (optical field D) of excitation light and fluorescence L_{f}. Examples of the dielectric 16 are polystyrene, SiO₂ and the like.

Further, the fluorescent dye molecule f is not particularly limited. The fluorescent dye molecule f may be a molecule of an organic dye or an inorganic dye, such as a quantum dot. In bio-measurement a sample that generates autofluorescence (absorption) is a measurement target in some cases. Therefore, it is desirable to use a fluorescent dye that generates fluorescence in a long wavelength range. Examples of infrared-excitable fluorescent dye are Hayashibara Biochemical Lab., Inc., NK-529 (excitation wavelength˜640 nm), NK-1836 (excitation wavelength˜650 nm), NK-2014 (excitation wavelength˜780 nm), Dyomics GmbH, DY-780-L (excitation wavelength˜760 nm), DY-785-L (excitation wavelength˜770 nm), and the like.

The fluorescent label (fluorescent substance) F is not particularly limited as long as the fluorescent label F includes a plurality of fluorescent dye molecules f that can be excited at a desirable wavelength and the plurality of fluorescent dye molecules f are enclosed by the dielectric 16 that transmits fluorescence L_{f} output from the fluorescent dye molecules f by excitation with evanescent light (optical field D).

For example, the fluorescent label (fluorescent substance) F may be produced, for example, as follows:

First, polystyrene particles (Estapor, Φ500 nm, 10% solid, carboxyl group, product No. K1-050) are prepared to obtain 0.1% solid in phosphate (polystyrene solution: pH7.0)

Next, an acetic acid ethyl solution (1 mL) containing 0.3 mg of fluorescent dye molecules (Hayashibara Biochemical Labs., Inc., NK-2014 (excitation˜780 nm) is produced.

Further, the polystyrene solution and the fluorescent dye solution are mixed together, and impregnated while the mixture evaporates. After then, centrifugation (15000 rpm, 4° C., 20 minutes, twice) is performed, and the supernatant is removed. Accordingly, fluorescent substance F, in which fluorescent dye is enclosed by polystyrene, is obtained. When the fluorescent substance F is produced by impregnating the fluorescent dye into the polystyrene particle through the aforementioned processes, the size or diameter of the particle of the fluorescent substance F is the same as that of polystyrene particle (Φ500 nm in the above example).

Alternatively, for example, commercially-available fluorescent beads, such as fluorescent beads produced by Bangs Laboratories, Inc. (product No. FC03F/8632, diameter of 510 nm, excitation wavelength of 660 nm, and fluorescence wavelength of 690 mm), fluorescent beads produced by Molecular Probes, Inc. (product No. F8807, diameter of 200 nm, excitation wavelength of 660 nm, fluorescence wavelength of 680 nm), fluorescent beads produced by Molecular Probes, Inc. (product No. F8816, diameter of 1000 nm, excitation wavelength of 625 m, fluorescence wavelength of 645 nm), and the like may be used.

When the fluorescent dye molecules are located too close to the metal layer, quenching occurs due to energy transfer to the metal. When the metal is a flat plane having a semi-infinite thickness, the magnitude (degree) of energy transfer is in inverse proportion to the cube of the distance. When the metal is a flat plane having a finitely thin thickness, the magnitude of energy transfer is in inverse proportion to the fourth power of the distance. Further, when the metal is microparticles, the magnitude of energy transfer is in inverse proportion to the sixth power of the distance. Therefore, it is desirable that the distance between the metal layer 12 and the fluorescent dye molecules f is at least a few nm, and it is more desirable that the distance is greater than or equal to 10 nm.

As described in the section of “Description of the Related Art”, conventionally, metal quenching has been prevented by making the fluorescent dye and the enhanced electric field generation surface on which the metal layer is exposed be apart from each other in such a manner that metal quenching does not occur. Alternatively, metal quenching has been prevented by providing a three-dimensional layer or coating, such as a dielectric layer (a metal oxide layer, a polymer layer or the like) or carboxymethyl dextran (CMD) coating, on the metal layer to make the fluorescent dye and the metal layer be apart from each other. However, formation of such layers or coatings on the metal layer to prevent metal quenching complicates the process of producing the sensor chip, and greatly increases the operation process. In contrast, when the fluorescent substance F, as described above, is used, it is possible to maintain at least a predetermined distance from the metal layer 12. Therefore, it is possible to effectively prevent metal quenching by using an extremely simple method.

In the present embodiment, the excitation light L₀ enters the interface between the dielectric plate 11 and the metal layer 12 in p-polarization to induce surface plasmons. The excitation-light irradiation optical system 20 includes an excitation light source 21 including a semiconductor laser (LD), which outputs the excitation light L₀. Further, the excitation-light irradiation optical system 20 includes a polarization adjustment device 23 that makes the excitation light L₀ linearly polarized (p-polarized), and a prism 22 arranged in such a manner that a surface of the prism 22 contacts the dielectric plate 11. The prism 22 guides the excitation light L₀ into the dielectric plate 11 so that the excitation light L₀ totally reflects at the interface between the dielectric plate 11 and the metal layer 12.

The polarization adjustment device 23 is a polarizer that extracts only p-polarized light. When light output from the excitation light source 21 is mainly p-polarized, it is not necessary that the polarization adjustment device 23 is provided. The material of the prism 22 is not particularly limited. It is desirable that glass, polymethyl methacrylate (PMMA), polycarbonate (PC), amorphous polyolefins including cyclo-olefin, or the like is used as the material of the prism 22.

In the present embodiment, the prism 22 and the dielectric plate 11 are in contact with each other through refractive-index-matching oil. The excitation light source 21 is arranged in such a manner that the excitation light L₀ enters the prism from another surface of the prism 22, which is different from the surface in contact with the dielectric plate 11, and enters a sample-contact-surface 10a of the sensor chip 10 at an angle greater than or equal to a total reflection angle. Further, the excitation light source 21 is arranged in such a manner that the excitation light L₀ enters the metal layer 12 at a specific angle that generates surface plasmon resonance. Further, a light guide member may be arranged between the excitation light source 21 and the prism 22, if necessary. Further, the prism 22 and the dielectric plate 11 may be formed in one piece.

The housing unit 13 is structured in such a manner that when the housing unit 13 houses the sensor chip 10, the sensor portion 14 of the sensor chip 10 is arranged on the prism 22, and that the light detection means 30 can detect fluorescence. The material of the housing unit 13 is not particularly limited as long as the material can transmit excitation light L₀ and signal light (fluorescence) L_{f}. Examples of the material of the housing unit 13 are inorganic oxide thin-film of SiO₂, TiO₂, HfO₂ or the like, organic polymers, such as polystyrene and PMMA, and the like. The cell (sensor chip 10) in the housing unit 13 is changeable.

The fluorescence (signal light) L_{f} is detected by the light detection means 30 from one surface side of the housing unit 13 (upper side in FIG. 1), the one surface side being opposite to the prism 22 side. A photodetector, such as CCD, PD (photodiode), photomultiplier and c-MOS, may be appropriately used as the light detection means 30. For example, LAS-1000 plus (product name), produced by FUJIFILM Corporation may optionally be used.

Next, a fluorescence detection method according to the present embodiment using the fluorescence detection apparatus 1 will be described.

Here, a case in which antigen A is detected as a detection target substance contained in sample S will be described as an example.

As the sensor chip 10, a sensor chip in which a metal film (metal layer) 12 is modified with fragmented antibody B₁ (F(ab′)₂ fragment) as an immobilization layer is prepared. The fragmented antibody B₁ is a primary antibody that specifically binds to the antigen A.

First, liquid sample S, which is an examination object (assay object or target), is poured into the housing unit 13 to make the liquid sample S contact with the metal film 12 of the sensor chip 10. Next, a solution containing fluorescent-label binding substance (labeling secondary antibody) B_{F} is poured into the housing unit 13 in a similar manner. The fluorescent-label binding substance B_{F} includes secondary antibody B₂, which is a second binding substance that specifically binds to the antigen A, and fluorescent label F. The fluorescent label F includes a plurality of fluorescent dye molecules f enclosed by the dielectric 16. In this case, the fragmented antibody B₁ that is used for surface modification of the metal film 12 and the secondary antibody B₂ of the fluorescent-label binding substance B_{F} are selected so that they bind to different sites of the antigen A, which is the detection target substance.

When the antigen A is present in the sample S, the antigen A specifically binds to the fragmented primary antibody B₁, and the secondary antibody B₂ in the fluorescent-label binding substance B_{F} binds to the antigen A. Consequently, a bound body of (fragmented primary antibody B₁)-(antigen A)-(secondary antibody B₂) (hereinafter, referred to as a sandwich bound body) is formed.

After then, unreacted fluorescent-label binding substance B_{F} is eliminated by using remaining liquid sample S or by separately pouring a buffer solution into the housing unit 13. Consequently, the sandwich bound body and the unreacted fluorescent-label binding substance B_{F} are separated from each other.

Timing of labeling the detection target substance (antigen A) is not particularly limited. Fluorescent label F may be added to the sample in advance before the detection target substance (antigen A) binds to the fragmented primary antibody B₁.

After then, the excitation light irradiation optical system 20 outputs excitation light L₀ toward a predetermined area of the dielectric plate 11 of the sensor chip 10. When the excitation light irradiation optical system 20 causes the excitation light L₀ to enter the interface between the dielectric plate 11 and the metal film 12 at a specific incident angle greater than or equal to a total reflection angle, evanescent waves extend into the sample S on the metal film 12. Further, the evanescent waves excite surface plasmons in the metal film 12. An optical field (electric field induced by evanescent waves) that has been generated on the metal film 12 by excitation light L₀ that has entered the interface is enhanced by the surface plasmons. Consequently, an enhanced optical field D is formed on the metal film 12. In the enhanced optical field D, and particularly in the vicinity of the surface of the metal film 12, the fluorescent label F is excited (substantially, the fluorescent dye molecules f in the fluorescent substance are excited), and fluorescence L_{f} is generated. The fluorescence is excited by the optical field enhancement effect by the surface plasmons.

It is possible to detect presence and/or the amount of the detection target substance that has bound to the fluorescent-label binding substance by detecting fluorescence by the light detection means 30.

The step of forming the sandwich bound body on the sensor portion and separating the sandwich bound body and the unreacted fluorescent-label binding substance B_{F} from each other may be performed before the sensor chip 10 is set in the housing unit of the detection apparatus 1. Alternatively, the process may be performed after setting the sensor chip 10.

In the aforementioned detection method of the present invention, a fluorescent substance F including a plurality of fluorescent dye molecules f enclosed by a dielectric 16 that transmits fluorescence output from the fluorescent dye molecules f is used as a fluorescent label of the fluorescent-label binding substance B_{F}. The fluorescent-label binding substance B_{F} in an amount corresponding to the detection target substance A contained in the sample S binds to the sensor portion 14. Since the fluorescent substance F includes the plurality of fluorescent dye molecules f within the substance, the number of the fluorescent dye molecules is large. Therefore, it is possible to obtain strong fluorescence. Further, since the fluorescent substance per se has a metal quenching prevention function. Therefore, it is possible to make the fluorescent substance F be located as close to the enhanced field generation surface (surface of the metal layer 12) of the sensor portion 14 as possible.

In the detection method of the present invention, the fluorescent-label binding substance B_{F} binds to the sensor portion 14 through the fragmented antibody B₁. Therefore, the distance between the fluorescent label F and the enhanced field generation surface (surface of the metal layer 2) is shorter, compared with a case in which the fluorescent-label binding substance B_{F} binds to the sensor portion 14 through an ordinary antibody (a full antibody molecule, which is not fragmented). Further, the fragmented antibody has a small number of three-dimensional obstacles. Especially when a disulfide bond and/or a thiol group has been exposed by fragmentation, the antigenic determinant group tends to be oriented to the sample side. Hence, the density of immobilized antibodies becomes high, and non-specific adsorption is reduced.

Therefore, according to the present invention, it is possible to effectively prevent metal quenching. Further, it is possible to efficiently utilize enhanced electric field to directly or indirectly detect fluorescent at high sensitivity.

### Design Modification Example of First Embodiment

In each of the aforementioned embodiments, the excitation light L₀ is collimated light that enters the interface at predetermined angle θ. Alternatively, the excitation light may be a fan beam (condensed light), as schematically illustrated in FIG. 4, which has angle width Δθ with respect to angle θ. When the excitation light is a fan beam, the excitation light enters the interface between the prism 122 and the metal film 112 on the prism 122 at an incident angle within the range of θ−Δθ/2 to θ+Δθ/2. When a resonance angle is present in the range of angles, it is possible to excite surface plasmons in the metal film 112. Further, the refractive index of the medium on the metal film changes when the sample is supplied onto the metal film. In other words, the refractive index of the medium before supply of the sample and the refractive index of the medium after supply of the sample differ from each other. Therefore, the resonance angle at which surface plasmons are generated changes. When the collimated light is used as the excitation light as in the aforementioned embodiments, it is necessary to adjust the incident angle of the collimated light every time when the resonance angle changes. However, as illustrated in FIG. 4, when the fan beam the incident angle of which has a certain width is used, it is possible to cope with the change in the resonance angle without adjusting the incident angle. Further, it is desirable that the fan beam has flat distribution, in which a change in the intensity of light according to the incident angle is small.

### Second Embodiment

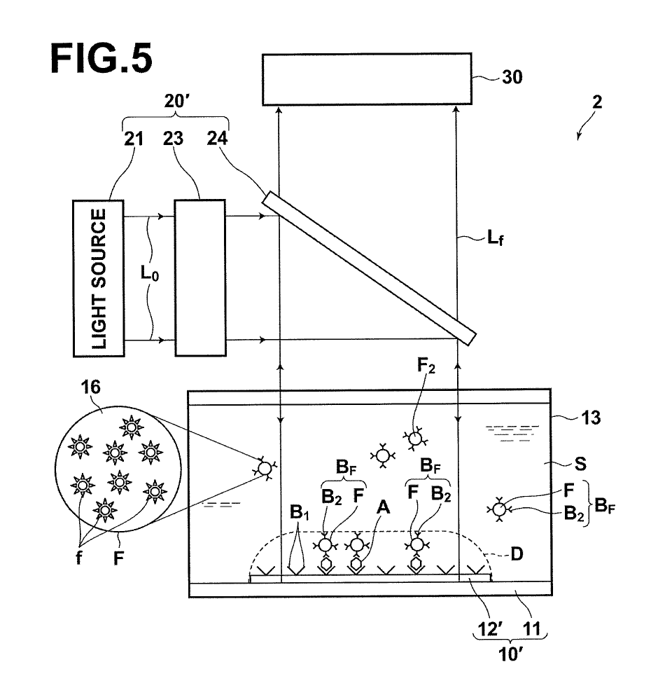

A detection method and apparatus according to a second embodiment will be described with reference to FIG. 5. FIG. 5 is a schematic diagram illustrating the structure of the whole detection apparatus of the second embodiment. The detection method and apparatus in this embodiment enhances an optical field by localized plasmon resonance, and detects fluorescence excited in the enhanced optical field. In the following descriptions, the same reference numerals will be assigned to elements corresponding to the elements in the first embodiment.

In a fluorescence detection apparatus 2 illustrated in FIG. 5, a sensor chip 10′ and an excitation light irradiation optical system 20′ differ from the elements of the fluorescence detection apparatus 1 of the first embodiment.

The sensor chip 10′ includes, as a metal layer 12′ provided on the dielectric plate 11, a metal fine structure body or a plurality of metal nanorods, which generate so-called localized plasmons by irradiation with the excitation light. The metal fine structure body includes an uneven structure (an uneven pattern, or projections/depressions) that is smaller than the wavelength of the excitation light L₀ on the surface thereof. Further, the size of each of the plurality of metal nanorods is smaller than the wavelength of the excitation light L₀. When the metal layer 12′ as described above, which generates localized plasmons, is provided, it is not necessary that the excitation light enters the interface between the metal layer 12′ and the dielectric plate 11 at a total reflection angle. Therefore, an excitation light irradiation optical system 20′ is arranged in such a manner that the excitation light L₀ is output to the sensor chip 10′ from the upper side of the dielectric plate 11.

The excitation light irradiation optical system 20′ includes a light source 21, such as a semiconductor laser (LD), a polarization adjustment device 23, and a half mirror 24. The light source 21 outputs the excitation light L₀, and the polarization adjustment device 23 makes the excitation light L₀ linearly polarized (p-polarized). The half mirror 24 reflects the excitation light L₀, and guides the excitation light L₀ to the sensor chip 10′. The half mirror 24 reflects the excitation light L₀, and transmits fluorescence L_{f}.

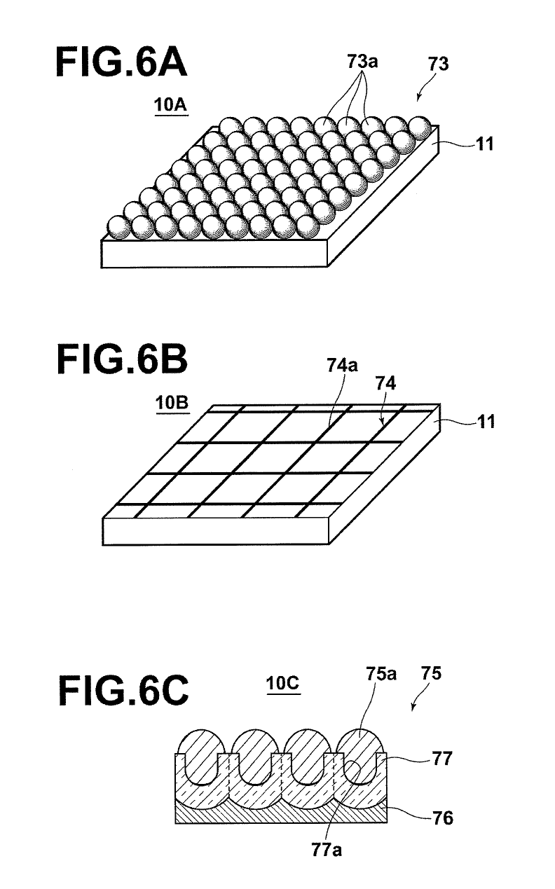

A specific example of the sensor chip 10′ will be described with reference to perspective views illustrated in FIGS. 6A through 6C.

A sensor chip 10A illustrated in FIG. 6A includes the dielectric plate 11 and a metal fine structure body 73. The metal fine structure body 73 includes a plurality of metal particles 73a fixed in array form on a predetermined area of the dielectric plate 11. The arrangement pattern of the metal particles 73a may be appropriately designed, and it is desirable that the arrangement pattern is substantially regular. This structure is designed in such a manner that an average particle diameter of the metal particles 73a and an average pitch thereof are smaller than the wavelength of the excitation light L₀.

A sensor chip 10B illustrated in FIG. 6B includes the dielectric plate 11 and a metal fine structure body 74 provided on a predetermined area of the dielectric plate 11. The metal fine structure body 74 is formed by a metal pattern layer. In the metal pattern layer, metal thin wires 74a are arranged in grid form by pattern formation. The pattern of the metal pattern layer may be appropriately designed, and it is desirable that the pattern is substantially regular. This structure is designed in such a manner that an average width (line width) of the metal thin wires 74a and an average pitch thereof are smaller than the wavelength of the excitation light L₀.

A sensor chip 10C illustrated in FIG. 6C includes a metal fine structure body 75 as disclosed in U.S. Patent Application Publication No. 20070158549. The metal fine structure body 75 includes a plurality of mushroom-shape metals 75a that have grown in a plurality of minute pores (holes) 77a in a metal oxide object 77. The minute pores 77a are formed in the process of anodic oxidation of a metal 76, such as Al. Here, the metal oxide object 77 corresponds to the dielectric plate 11. The metal fine structure body 75 can be produced by obtaining a metal oxide object (Al₂O₃ or the like) by performing anodic oxidation on a part of a metal body (Al or the like) and by making the metal 75a grow in each of the plurality of minute pores 77a in the metal oxide object 77 by plating or the like. The plurality of minute pores 77a are formed in the process of anodic oxidation.

In the example illustrated in FIG. 6C, the top portion of the mushroom-shape metal 75a has particle form. Therefore, when the metal fine structure body 75 is observed from the surface of the sample plate, the metal fine structure body 75 is structured in such a manner that metal microparticles are arranged. In this structure, the top portions of the mushroom-shape metals 75a are projections (projections in an uneven pattern), and the structure is designed in such a manner that an average diameter of the projections (top portions) and an average pitch thereof are smaller than the wavelength of excitation light L₀.

Further, as the metal layer 12′, which generates localized plasmons by irradiation with excitation light, various kinds of other metal fine structure bodies may be used. The various kinds of metal fine structure bodies utilize fine structures obtained by performing anodic oxidation on metal bodies, as disclosed in U.S. Patent Application Publication No. 20060234396, U.S. Patent Application Publication No. 20060181701, and the like.

Further, the metal layer that generates localized plasmons may be formed by a metal film (coating) the surface of which has been coarsened. As a method for coarsening the surface, there is an electro-chemical method utilizing oxidation/reduction or the like. Further, the metal layer may include a plurality of metal nanorods arranged on a sample plate. The short-axial length of the metal nanorods is approximately 3 nm to 50 nm, and the long-axial length of the metal nanorods is approximately 25 nm to 1000 nm. Further, the long-axial length should be less than the wavelength of the excitation light. The metal nanorods are disclosed, for example, in U.S. Patent Application Publication No. 20070118936, or the like.

Further, it is desirable that the metal fine structure body and the metal nanorods, which are used as the metal layer 12′, contain, as a main component, at least one kind of metal selected from the group consisting of Au, Ag, Cu, Al, Pt, Ni, Ti and alloys thereof.

In the present embodiment, the fragmented primary antibody B₁ has directly bound to the surface of the metal layer 12′ in a manner similar to the first embodiment. In the present embodiment, desirable material of each element of the sensor chip 10′ is similar to the first embodiment. Further, except for the metal layer 12′, a desirable thickness of each element is similar to the first embodiment.

A fluorescence detection method of the present embodiment using the fluorescence detection apparatus 2 will be described.

The processes of preparing a sensor chip and causing antigen-antibody reaction are similar to the processes in the first embodiment. Therefore, the explanation about these processes will be omitted in the following embodiments.

In a state in which the sensor chip 10′ is set in the housing unit 13, excitation light L₀ is output to a predetermined area on the dielectric plate 11 of the sensor chip 10′ from the excitation light irradiation optical system 20′. The excitation light L₀ output from the light source 21 is reflected by a half mirror 24, and enters the sample-contact surface of the sensor chip 10′. Consequently, localized plasmons are excited on the surface of the metal layer 12′ by irradiation with the excitation light L₀. Further, the optical field (an electric field induced by near field light) that has been generated on the metal layer 12′ by the incident excitation light L₀ is enhanced by the localized plasmons. Accordingly, enhanced optical field D is formed on the metal layer. In the enhanced electric field D, particularly in the vicinity of the surface of the metal film 12′, the fluorescent substance F is excited (fluorescent dye molecules f in the fluorescent substance are substantially excited), and fluorescence L_{f} is output. At this time, the fluorescence is enhanced by the optical field enhancement effect by the localized plasmons. The fluorescence L_{f} is detected by the light detection means 30. Accordingly, it is possible to detect presence and/or the amount of the detection target substance that has bound to the fluorescent-label binding substance by detecting fluorescence at the light detection means 30.

In the present embodiment, the fluorescent substance F including a plurality of fluorescent dye molecules f enclosed by a dielectric that transmits fluorescence output from the fluorescent dye molecules f is used in a manner similar to the first embodiment. Further, the fluorescent-label binding substance B_{F} binds to the surface of the sensor portion 14 through the fragmented antibody B₁. Therefore, it is possible to achieve an advantageous effect similar to the first embodiment.

### Third Embodiment

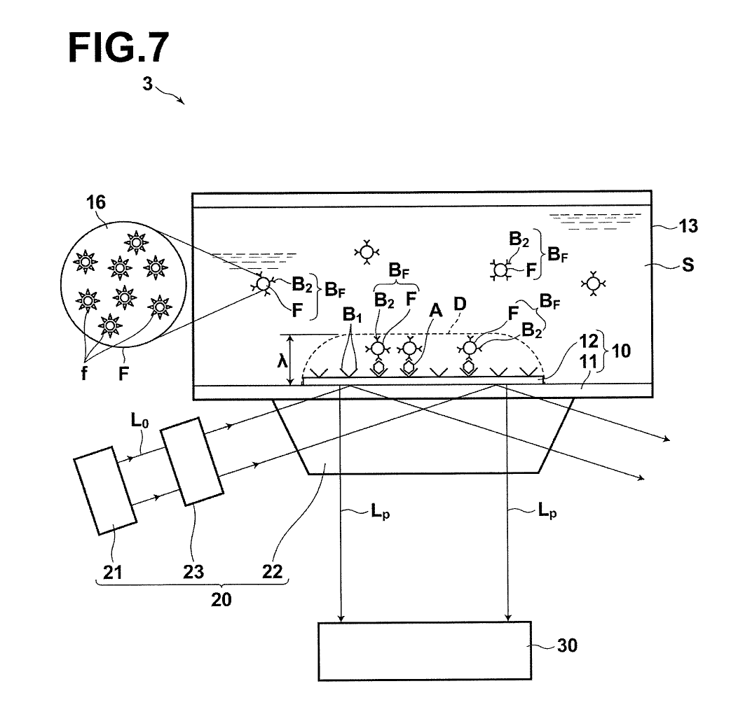

A detection method and apparatus according to a third embodiment will be described with reference to FIG. 7. FIG. 7 is a schematic diagram illustrating the structure of the detection apparatus of the third embodiment. The detection method and apparatus in this embodiment enhances an electric field by surface plasmon resonance, and fluorescence excited in the enhanced electric field newly induces plasmons in the metal layer. Further, light from the newly-induced plasmons radiates from the opposite surface of the dielectric plate, the opposite surface being opposite to the metal-layer-formation surface of the dielectric plate. Further, radiation light from the newly induced plasmons is detected in the radiation light detection method and apparatus of this embodiment.

In a radiation light detection apparatus 3, illustrated in FIG. 7, the arrangement of the light detection means 30 is different from the arrangement of the light detection means 30 in the fluorescence detection apparatus of the first embodiment. In the radiation light detection apparatus 3 of the present embodiment, the light detection means 30 is arranged in such a manner that radiation light Lₚ from the newly induced plasmons is detected. The plasmons are newly induced in the metal layer 12 by fluorescence that has been excited in the enhanced electric field, and the radiation light Lₚ from the newly induced plasmons radiates from the opposite surface of the dielectric plate, the opposite surface (lower side in FIG. 7) being opposite to the metal layer formation surface of the dielectric plate 11.

A radiation light detection method of the present embodiment using the radiation light detection apparatus 3 will be described.

In a state in which the sensor chip 10 is set in the housing unit 13, excitation light L₀ is output from the excitation light irradiation optical system 20 in a manner similar to the first embodiment. The excitation light irradiation optical system 20 outputs the excitation light L₀ in such a manner that the excitation light L₀ enters the interface between the dielectric plate 11 and the metal layer 12 at a specific incident angle that is greater than or equal to a total reflection angle. When the excitation light L₀ enters the interface in such a manner, evanescent waves extend to the sample S on the metal layer 12, and surface plasmons are excited in the metal layer 12 by the evanescent waves. Further, the optical field (an electric field induced by evanescent waves) that has been generated on the metal layer by the incident excitation light L₀ is enhanced by the surface plasmons. Accordingly, enhanced optical field D is formed on the metal layer. In the enhanced electric field D, particularly in the vicinity of the surface of the metal film 12′, the fluorescent substance F is excited (fluorescent dye molecules f in the fluorescent substance are substantially excited), and fluorescence L_{f} is output. At this time, the fluorescence is enhanced by the optical field enhancement effect by the surface plasmons. The fluorescence L_{f} generated on the metal film 12 newly induces surface plasmons in the metal film 12, and radiation light Lₚ is output by the surface plasmons at a specific angle from an opposite surface of the sensor chip 10, the opposite surface being opposite to the metal film formation surface of the sensor chip 10. Further, the radiation light Lₚ is detected by the light detection means 30. Accordingly, it is possible to detect presence and/or the amount of the detection target substance that has bound to the fluorescent-label binding substance B_{F}.

The radiation light Lₚ is generated when fluorescence couples to surface plasmons of a specific wavenumber in the metal film. The wavenumber of the surface plasmons that couple to the fluorescence is determined by the wavelength of the fluorescence. Further, the output angle of radiation light is determined by the wavenumber. Ordinarily, the wavelength of excitation light L₀ and the wavelength of fluorescence differ from each other. Therefore, the wavenumber of the surface plasmons excited by the fluorescence differs from the wavenumber of the surface plasmons generated by the excitation light L₀. Therefore, the radiation light Lₚ is output at an angle that is different from the incident angle of the excitation light L₀.

In the present embodiment, the fluorescent substance F including a plurality of fluorescent dye molecules f enclosed by a dielectric that transmits fluorescence from the fluorescent dye molecules f is used in a manner similar to the first embodiment. Further, the fluorescent-label binding substance B_{F} binds to the surface of the sensor portion 14 through the fragmented antibody B₁. Therefore, it is possible to achieve an advantageous effect similar to the first embodiment.

Further, in the present embodiment, light induced by the fluorescence generated on the surface (front surface) of the sensor is detected from the back side of the sensor. Therefore, it is possible to reduce the distance of a solvent through which the fluorescence L_{f} passes (travels), and which greatly absorbs light, to approximately several tens nm. Therefore, it is possible to substantially ignore light absorption, for example, in blood. Therefore, it is possible to perform measurement without performing pre-processing, such as centrifuging the blood to remove a colored component, such as red blood cells, from the blood, and filtering the blood through a blood cell filter to obtain blood serum or blood plasma.

### Fourth Embodiment

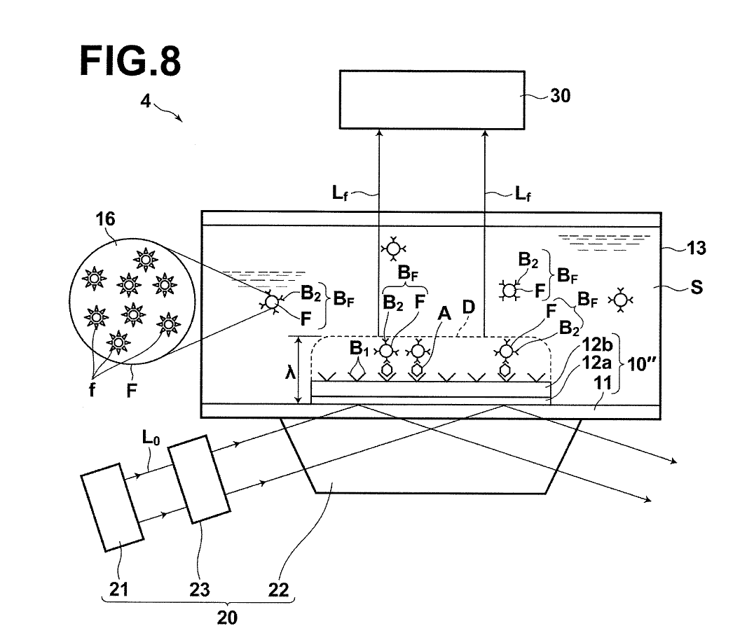

A detection method and apparatus according to a fourth embodiment will be described with reference to FIG. 8. FIG. 8 is a schematic diagram illustrating the structure of the whole detection apparatus of the fourth embodiment. The detection method and apparatus in this embodiment uses a sensor chip including an optical waveguide layer on the metal layer, and excites an optical waveguide mode in the optical waveguide layer by irradiation with excitation light. Further, an optical field is enhanced by the optical waveguide mode, and fluorescence is excited in the enhanced optical field to detect the fluorescence.

The structure of the fluorescence detection apparatus 4 illustrated in FIG. 8 is the same as the structure of the fluorescence detection apparatus of the first embodiment. However, the sensor chip used in the present embodiment differs from the sensor chip used in the first embodiment. The mechanism of enhancing the optical field in the present embodiment differs from the first embodiment because of the difference in the sensor chip.

A sensor chip 10″ includes an optical waveguide layer 12b on the metal layer 12a. The thickness of the optical waveguide layer 12b is not particularly limited. The thickness of the optical waveguide layer 12b may be determined so that the optical waveguide mode is induced. The thickness is determined by considering the wavelength of the excitation light L₀, the incident angle of the excitation light L₀, the refractive index of the optical waveguide layer 12b, and the like. For example, when a laser beam that has a center wavelength of 780 nm is used as the excitation light L₀ in a manner similar to the aforementioned embodiment, and the optical waveguide layer 12b made of a single layer of silicon oxide film is used, it is desirable that the thickness of the optical waveguide layer 12b is approximately in the range of 500 to 600 nm. Further, the optical waveguide layer 12b may have layered structure including at least a layer of internal optical waveguide layer made of an optical waveguide material, such as a dielectric. It is desirable that the layered structure is an alternately-layered structure in which an internal optical guide layer and an internal metal layer are sequentially deposited from the metal layer side.

In the sensor chip 10″, when the surface (top-surface) of the optical waveguide layer 12b is a metal layer, if the fluorescent dye molecules f are located close to the surface of the optical waveguide layer 12b directly, metal quenching occurs as described in the first embodiment. Therefore, it is possible to achieve an effect similar to the first embodiment by directly binding the fragmented primary antibody B1 to the top surface of the optical waveguide layer 12b, and by using, as a fluorescent label, a fluorescent substance F including a plurality of fluorescent dye molecules F enclosed by a dielectric 16 that transmits fluorescence L_{f} excited by evanescent light (optical field D) and output from the plurality of fluorescent dye molecules f. Further, in the present embodiment, desirable material of each element of the sensor chip 10″ is similar to the first embodiment. Further, except for the metal layer 12a, a desirable thickness of each element is similar to the first embodiment.

A fluorescence detection method according to the present embodiment using the fluorescence detection apparatus 4 will be described.

In a state in which the sensor chip 10″ is set in the housing unit 13, excitation light L₀ is output from the excitation light irradiation optical system 20 in a manner similar to the first embodiment. The excitation light irradiation optical system 20 outputs the excitation light L₀ in such a manner that the excitation light L₀ enters the interface between the dielectric plate 11 and the metal layer 12a at a specific incident angle that is greater than or equal to a total reflection angle. When the light L₀ enters the interface in such a manner, evanescent waves extend to the optical waveguide layer 12b on the metal layer 12a, and the evanescent waves couple to the optical waveguide mode of the optical waveguide layer 12b. Accordingly, an optical waveguide mode is excited. Further, the optical field (an electric field induced by evanescent waves) that has been generated on the metal layer by the incident excitation light is enhanced by the optical waveguide mode. Accordingly, enhanced optical field D is formed on the optical waveguide layer. In the enhanced electric field D, particularly in the vicinity of the surface of the metal layer 12a, the fluorescent substance F is excited (fluorescent dye molecules f in the fluorescent substance are substantially excited), and fluorescence L_{f} is output. At this time, the fluorescence is enhanced by the optical field enhancement effect by the optical waveguide mode. The fluorescence L_{f} is detected by the light detection means 30. Accordingly, it is possible to detect presence and/or the amount of the detection target substance that has bound to the fluorescent-label binding substance.

In the present embodiment, the fluorescent substance F including a plurality of fluorescent dye molecules f enclosed by a dielectric that transmits fluorescence from the fluorescent dye molecules f is used in a manner similar to the first embodiment. Further, the fluorescent-label binding substance B_{F} binds to the surface of the sensor portion 14 through the fragmented antibody B₁. Therefore, it is possible to achieve an advantageous effect similar to the first embodiment.

Further, in the distribution of optical field enhanced by excitation of the optical waveguide mode, the degree of attenuation of the electric field according to the distance from the surface is small, compared with the degree of attenuation in the distribution of optical field generated by surface plasmons. Therefore, when a fluorescent substance that has a large diameter, and which includes a plurality of fluorescent dye molecules, is used as the fluorescent label, a greater fluorescent amount increase effect is achieved in enhancement of the optical field by the optical waveguide mode, compared with enhancement of the optical field by surface plasmons.

### Fifth Embodiment

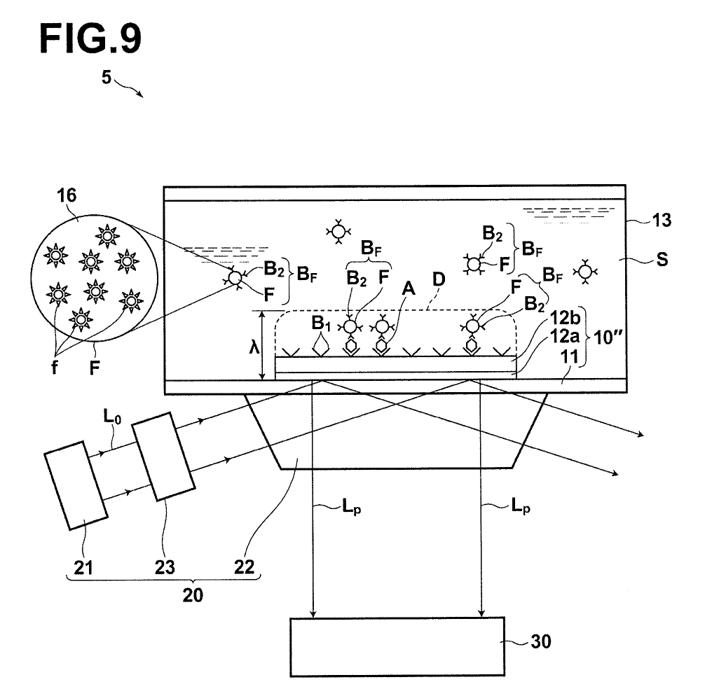

A detection method and apparatus according to a fifth embodiment will be described with reference to FIG. 9. FIG. 9 is a schematic diagram illustrating the structure of the whole detection apparatus of the fifth embodiment. The detection method and apparatus of this embodiment uses a sensor chip including an optical waveguide layer on the metal layer, and excites an optical waveguide mode in the optical waveguide layer by irradiation with excitation light. Further, an optical field is enhanced by the optical waveguide mode, and fluorescence excited in the enhanced optical field newly induces plasmons in the metal layer. Further, light from the newly-induced plasmons radiates from the opposite surface of the dielectric plate, the opposite surface being opposite to the metal-layer-formation surface of the dielectric plate. Further, radiation light from the newly induced plasmons is detected in the radiation light detection method and apparatus of this embodiment.

The structure of the radiation light detection apparatus 5 illustrated in FIG. 9 is similar to the radiation light detection apparatus of the third embodiment. The sensor chip used in the detection method of the present embodiment is similar to the sensor chip used in the fluorescence detection method of the fourth embodiment.

A radiation detection method according to the present embodiment using the radiation light detection apparatus 5 will be described.

In a state in which the sensor chip 10″ is set in the housing unit 13, excitation light L₀ is output from the excitation light irradiation optical system 20 in a manner similar to the first embodiment. The excitation light irradiation optical system 20 outputs the excitation light L₀ in such a manner that the excitation light L₀ enters the interface between the dielectric plate 11 and the metal layer 12a at a specific incident angle that is greater than or equal to a total reflection angle. When the light L₀ enters the interface in such a manner, evanescent waves extend to the optical guide layer 12b on the metal layer 12a, and the evanescent waves couple to the optical waveguide mode of the optical waveguide layer 12b. Accordingly, an optical waveguide mode is excited. Further, the optical field (an electric field induced by evanescent waves) that has been generated on the optical waveguide layer 12b by the incident excitation light is enhanced by the optical waveguide mode. Accordingly, enhanced optical field D is formed on the optical waveguide layer 12b. In the enhanced electric field D, particularly in the vicinity of the surface of the metal layer 12a, the fluorescent substance F is excited (fluorescent dye molecules f in the fluorescent substance are substantially excited), and fluorescence L_{f} is output. At this time, the fluorescence is enhanced by the optical field enhancement effect by the optical waveguide mode. The fluorescence L_{f} generated on the optical waveguide layer 12b newly induces surface plasmons in the metal film 12a, and radiation light Lₚ is output by the surface plasmons at a specific angle from an opposite surface of the sensor chip 10, the opposite surface being opposite to the metal film formation surface. Further, the radiation light Lₚ is detected by the light detection means 30. Accordingly, it is possible to detect presence and/or the amount of the detection target substance labeled with the fluorescent label F.

In the present embodiment, the fluorescent substance F including a plurality of fluorescent dye molecules f enclosed by a dielectric that transmits fluorescence from the fluorescent dye molecules f is used in a manner similar to the first embodiment. Further, the fluorescent-label binding substance B_{F} binds to the surface of the sensor portion 14 through the fragmented antibody B₁. Therefore, it is possible to achieve an advantageous effect similar to the first embodiment.

Further, in the present embodiment, light induced by the fluorescence generated on the surface (front surface) of the sensor is detected from the back side of the sensor. Therefore, it is possible to reduce the distance of a solvent through which the fluorescence L_{f} passes, and which greatly absorbs light, to approximately several tens nm. Hence, it is possible to achieve an effect similar to the third embodiment.

Further, since the electric field enhancement by excitation of the optical waveguide mode is used, it is possible to achieve a fluorescent amount increase effect similar to the fourth embodiment.

In the sensor chips 10″ in the fourth and fifth embodiments, when the top surface of the optical waveguide layer 12b is a dielectric layer, it is impossible to directly bind the fragmented antibody onto the surface of the optical waveguide layer 12b. In such a case, the top surface of the optical waveguide layer 12b needs to be processed, for example, by forming a self-assembled monolayer (SAM), and the fragmented primary antibody B₁ is bound to the surface after processing.

Further, in this case, even if the fluorescent dye molecules f are located close to the surface of the sensor chip, metal quenching does not occur. When the thickness of the dielectric layer on the top is sufficiently thick and fluorescent signals from the fluorescent dye molecules f are not significantly affected by the metal layer, a plurality of fluorescent dye molecules (light-responsive substance) f may be used as the fluorescent label instead of the fluorescent substance F. However, when a fluorescent substance F including a plurality of fluorescent dye molecules f enclosed by the dielectric 16 that transmits fluorescence L_{f} excited by evanescent light (optical field D) output from the plurality of fluorescent dye molecules f is used, the number of the fluorescent dye molecules that can be used for sensing is large. Since the plurality of fluorescent dye molecules f are enclosed in the dielectric 16, it is possible to obtain stronger fluorescence.

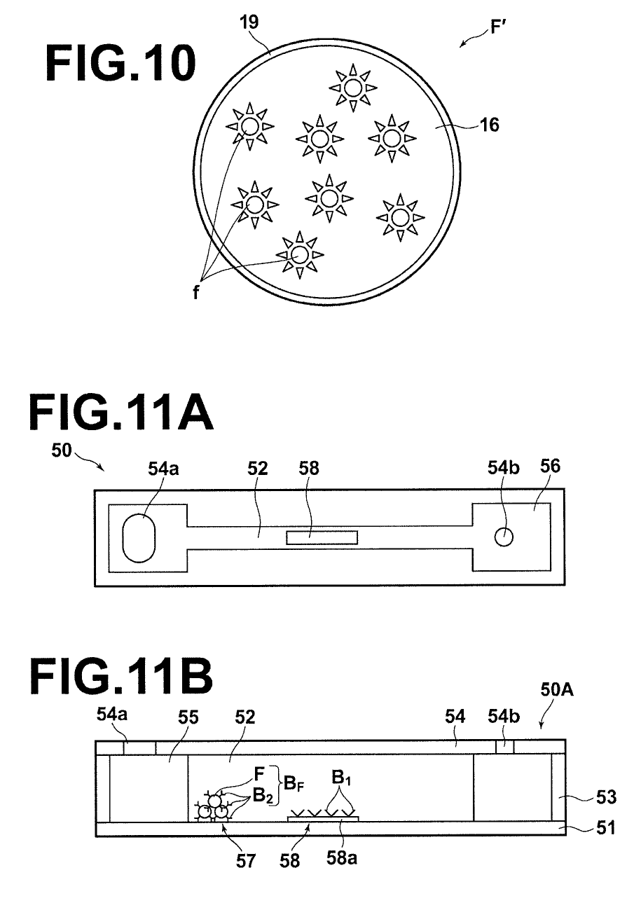

In the first through fifth embodiments, as illustrated in FIG. 10, fluorescent label F′ having a metal coating 19 provided on the surface of the dielectric 16 may be used. The thickness of the metal coating 19 should be sufficiently thin to transmit fluorescence. The metal coating 19 may be provided on the entire surface of the dielectric 16. Alternatively, the metal coating 19 may be provided in such a manner that a part of the surface of the dielectric 16 is exposed. As the material of the metal coating 19, a metal material similar to the aforementioned material of the metal layer may be used.

When the metal coating 19 is provided on the surface of the fluorescent substance, the surface plasmons or localized plasmons generated in the metal layers 12, 12′ of the sensor chips 10, 10′ couple to a whispering gallery mode of the metal coating 19 of the fluorescent substance F′. Therefore, it is possible to more efficiently excite the fluorescent dye molecules f in the fluorescent substance F′. The whispering gallery mode is an electromagnetic wave mode that is localized on the spherical surface of a micro-sphere of approximately Φ5300 nm or less, such as the fluorescent substance used here, and that travels around the spherical surface of the micro-sphere.

An example of a method for applying the metal coating to the fluorescent substance will be described.

First, an anti-metal-quenching fluorescent substance is produced through the aforementioned procedure. Further, the surface of the fluorescent substance is modified with polyethyleneimine (PEI) (EPOMIN, produced by NIPPON SHOKUBAI CO., LTD).

Next, Pd nano particles having a diameter of 15 nm (average particle diameter of 19 nm, produced by TOKURIKI-HONTEN) is adsorbed by the PEI on the surface of the particle.

The polystyrene particle that has adsorbed the Pd nano particles is impregnated in electroless gold plating solution (HAuCl₄, produced by Kojima Chemicals, Co., LTD.). Accordingly, a gold coating is deposited on the surface of the polystyrene particle by electroless plating using Pd nano particles as a catalyst.

### “Sample Cell for Detection”

A sample cell for detection that is used as a sensor chip in the detection method of the present invention will be described.

### Sample Cell According to First Embodiment

FIG. 11A is a plan view illustrating the structure of a sample cell 50A according to a first embodiment that is suitable for an assay by using a sandwich method. FIG. 11B is a sectional side view of the sample cell 50A.

The sample cell 50A for detection includes a base (substrate) 51, a spacer 53, and an upper plate 54. The base 51 is formed by a dielectric plate. The spacer 53 retains liquid sample S on the base 51, and forms a flow path 52 of the liquid sample S. The upper plate 54 is formed by a glass plate that has an injection opening 54a for injecting the sample S and an air hole 54b for discharging the sample that has flowed down through the flow path 52. Further, a sensor portion 58 including a metal layer 58a is provided in a predetermined area on the sample-contact surface of the base 51. The sensor portion 58 is provided between the injection opening 54a and the air hole 54b of the flow path 52. Further, a membrane filter 55 is provided in a portion of the sample cell 50 from the injection opening 54 to the flow path 52. Further, a waste liquid reservoir 56 is provided on the downstream side of the flow path 52. The waste liquid reservoir 56 is connected to the air hole 54b.

In the sample cell 50A of the present embodiment, a labeling secondary antibody adsorption area 57 and a measurement area 58 are sequentially formed on the base 51 from the upstream side of the flow path 52. In the labeling secondary antibody adsorption area 57, a fluorescent-label binding substance B_{F} (hereinafter, referred to as “labeling secondary antibody B_{F}”) has been physically adsorbed. The fluorescent-label binding substance B_{F} contains the secondary antibody (second binding substance) B₂ that specifically binds to the antigen, which is the detection target substance, and fluorescent substance F, the surface of which is modified with the secondary antibody B₂. In the measurement area 58, a primary antibody (first binding substance) B₁ is immobilized. The primary antibody B₁ specifically binds to the antigen, which is the detection target substance. The measurement area 58 corresponds to the sensor portion. In this example, only one measurement area is provided in the sensor portion. However, the number of measurement areas may be two or more.

In each measurement area 58 on the base 41, a gold (Au) film 58a is deposited as the metal layer. Further, the fragmented primary antibody B₁ is immobilized on the Au film 58a in the measurement area 58.

The sample cell 50A may be used instead of the sensor chip in any of the detection apparatuses and methods in the first through fifth embodiments.

In the sample cell 50A, another measurement area may be provided in such a manner that in the housing unit 13, the measurement area can move in X direction relative the excitation light irradiation optical system 20 and the detector 30. After the fluorescence or radiation light from the measurement area 58 is detected and measured, the detection position may be moved to detect fluorescence or radiation light from a second measurement area. Further, calibration with respect to fluctuation factors may be performed.

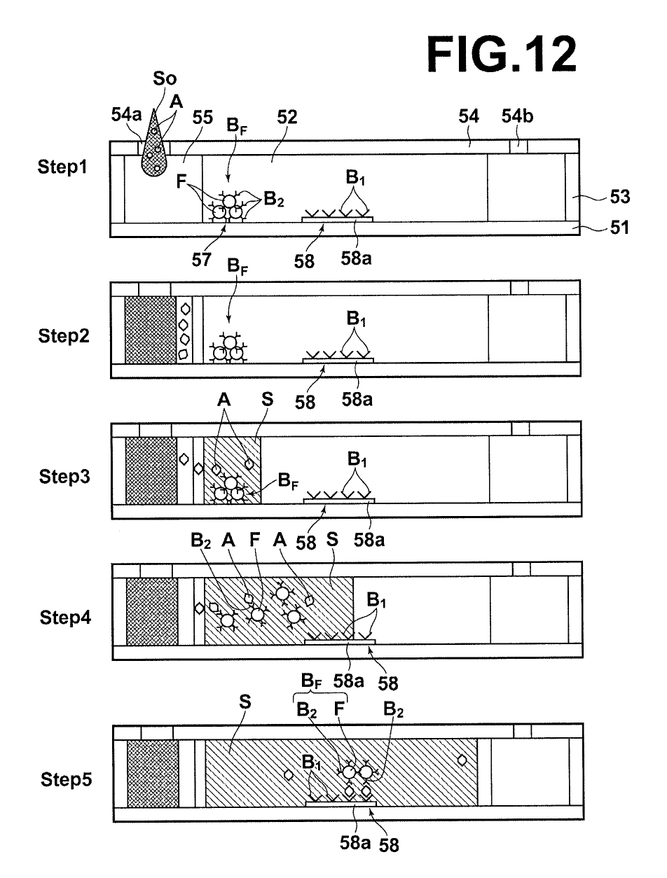

With reference to FIG. 12, assay procedures by using a sandwich method will be described. In the assay procedures, whether blood (whole blood) contains an antigen, which is a detection target substance, is detected by using the sample cell 50A for detection of the present embodiment.

Step 1: Blood (whole blood) Sₒ, which is the assay target (examination target), is injected from the injection opening 54a. Here, a case in which the antigen that is the detection target substance is included in the blood Sₒ will be described. In FIG. 12, the whole blood Sₒ is indicated by a mesh.

Step 2: The whole blood Sₒ is filtered by the membrane filter 55, and large molecules, such as erythrocyte (red blood cells) and leukocyte (white blood cells), remain as a residue.

Step 3: Blood (plasma, blood plasma) S after blood cells (blood corpuscles) are removed by the membrane filter 55 penetrates into the flow path 52 by a capillary action. Alternatively, a pump may be connected to the air hole 54b to accelerate reaction, thereby reducing detection time. The pump sucks the blood after blood cells are removed by the membrane filter 55 and pumps (pressures to discharge) the sucked blood, thereby causing the blood to flow down through the path. In FIG. 12, the blood plasma S is indicated by a shadow.

Step 4: The blood plasma S that has penetrated into the flow path 52 and the labeling secondary antibody B_{F} are mixed together. Accordingly, antigen A in the blood plasma and the secondary antibody B₂ of labeling secondary antibody B_{F} bind to each other.

Step 5: The blood plasma S gradually flows down to the air hole 54b side along the flow path 52. The antigen A that has bound to the secondary antibody B₂ binds to the fragmented primary antibody B₁ that has been immobilized in the first measurement area 58. Accordingly, a so-called sandwich is formed, in which the antigen A is sandwiched between the fragmented primary antibody B₁ and the secondary antibody B₂ (labeling secondary antibody B_{F}).

After steps 1 through 5, in which the blood is injected from the injection opening, a sandwich (an antigen A is sandwiched between a fragmented primary antibody B₁ and a labeling secondary antibody B_{F}) is formed on the measurement area 58, the intensity of fluorescence or radiation light (hereinafter, referred to as “signal”) from the measurement area 58 is detected.

In the present embodiment, the base 51 is made of a dielectric plate, and the dielectric plate also functions as the dielectric plate of the sensor portion. Alternatively, only a part of the base 51, the part in which the sensor portion is formed may be made of the dielectric plate.

The sample cell 50A may be used in any of the detection apparatuses and methods in the first through fifth embodiments.

### Sample Cell According to Second Embodiment

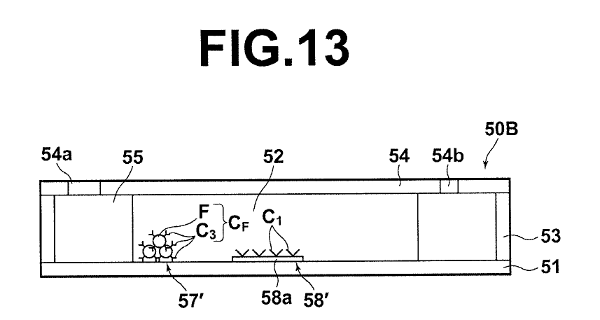

FIG. 13 is a sectional side view of a sample cell of the second embodiment that is suitable for an assay by a competition method. In a sample cell 50B of the present embodiment, a labeling secondary antibody adsorption area 57′ and a measurement area 58′ are sequentially formed on the base 51 of the sample cell 50B from the upstream side of the flow path 52. In the labeling secondary antibody adsorption area 57′, a fluorescent-label binding substance C_{F} (hereinafter, referred to as “labeling secondary antibody C_{F}”) has been physically adsorbed. The fluorescent-label binding substance C_{F} contains the secondary antibody (third binding substance) C₃ that does not bind to the antigen A, which is the detection target substance, and that specifically binds to the fragmented primary antibody, which will be described later. Further, the fluorescent-label binding substance C_{F} contains fluorescent substance the surface of which is modified with the secondary antibody C₃. In the measurement area 58′, a fragmented primary antibody (first binding substance) C₁ is immobilized. The fragmented primary antibody C₁ specifically binds to the antigen A, which is the detection target substance, and the secondary antibody C₃.

In the measurement area 58′, a gold (Au) film 58a, as a metal layer, is formed on the base 51. Further, the fragmented primary antibody C₁ is immobilized on the gold layer 58a in the measurement area 58′.

The antigen A and the secondary antibody C₃ competitively bind to the fragmented primary antibody C₁ immobilized in the measurement area 58′.

The sample cell SOB may be used instead of the sensor chip in any of the detection apparatuses and methods of the first through fifth embodiments in a manner similar to the sample cell 50A.

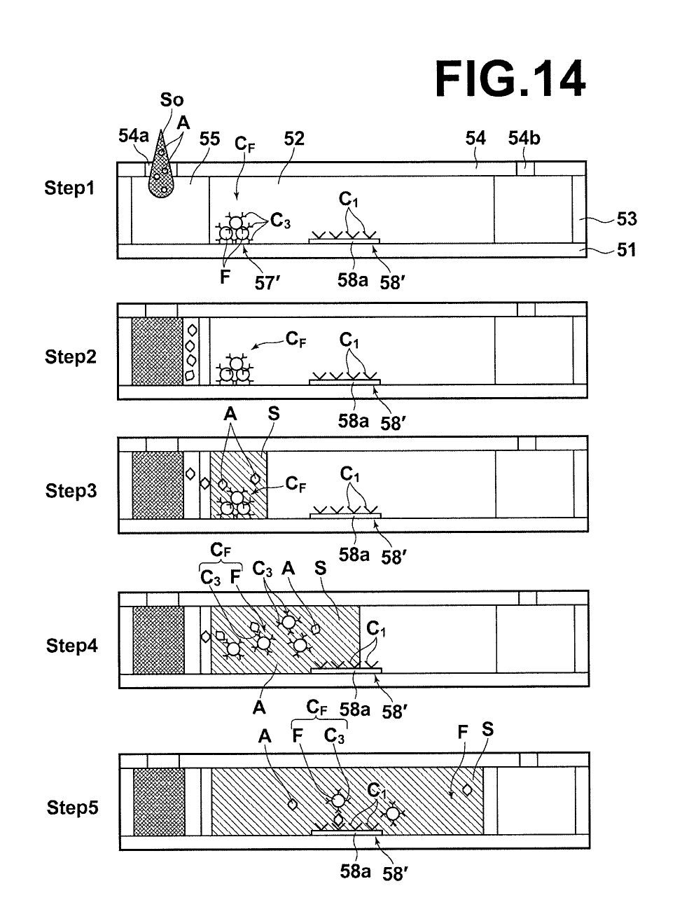

With reference to FIG. 14, assay procedures by using a competition method will be described. In the assay procedures, the sample cell 50B for detection of the present embodiment is used, and an assay is performed as to whether an antigen, which is a detection target substance, is included in the blood (whole blood).

Step 1: Blood (whole blood) Sₒ, which is the assay target, is injected from an injection opening 54a. Here, a case in which an antigen that is the detection target substance is included in the blood Sₒ will be described. In FIG. 14, the whole blood Sₒ is indicated by a mesh.

Step 2: The whole blood Sₒ is filtered by a membrane filter 55, and large molecules, such as erythrocyte (red blood cells) and leukocyte (white blood cells), remain as the residue.

Step 3: The blood (plasma, blood plasma) S after blood cells are removed by the membrane filter 55 penetrates into the flow path 52 by a capillary action. Alternatively, a pump may be connected to the air hole 54b to accelerate reaction, thereby reducing detection time. The pump sucks the blood after blood cells are removed by the membrane filter 55 and pumps the sucked blood, thereby causing the blood to flow down in the path. In FIG. 14, the blood plasma S is indicated by a shadow.

Step 4: The blood plasma S that has penetrated into the flow path 52 and the labeling secondary antibody C_{F} are mixed together.

Step 5: The blood plasma S gradually flows down to the air hole 54b side along the flow path 52. The antigen A and the secondary antibody C₃ of the labeling secondary antibody C_{F} competitively bind to the fragmented primary antibody C₁ that has been immobilized on the measurement area 58′.

As described above, in Steps 1 through 5, the blood is injected from the injection opening and the antigen A and the secondary antibody C₃ competitively bind to the fragmented primary antibody C₁ on the first measurement area 58′. After Steps 1 through 5, signals, such as the intensity of the fluorescence or radiation light, from the measurement area 58′ are detected.

In the competition method, when the concentration of the detection target substance A is higher, the amount of the third bonding substance C₃ that binds to the first binding substance C₁ decreases. Specifically, since the number of particles of the fluorescent substance F on the metal layer becomes smaller, the intensity of fluorescence becomes lower. In contrast, when the concentration of the detection target substance A is lower, the amount of the third bonding substance C₃ that binds to the first binding substance C₁ increases. Specifically, since the number of particles of the fluorescent substance F on the metal layer becomes larger, the intensity of fluorescence becomes higher. In the competition method, measurement is possible if at least one epitope is present in the detection target substance. Therefore, the competition method is suitable to detect a substance that has low molecular weight.

### (Design Modification Example of Sample Cell)

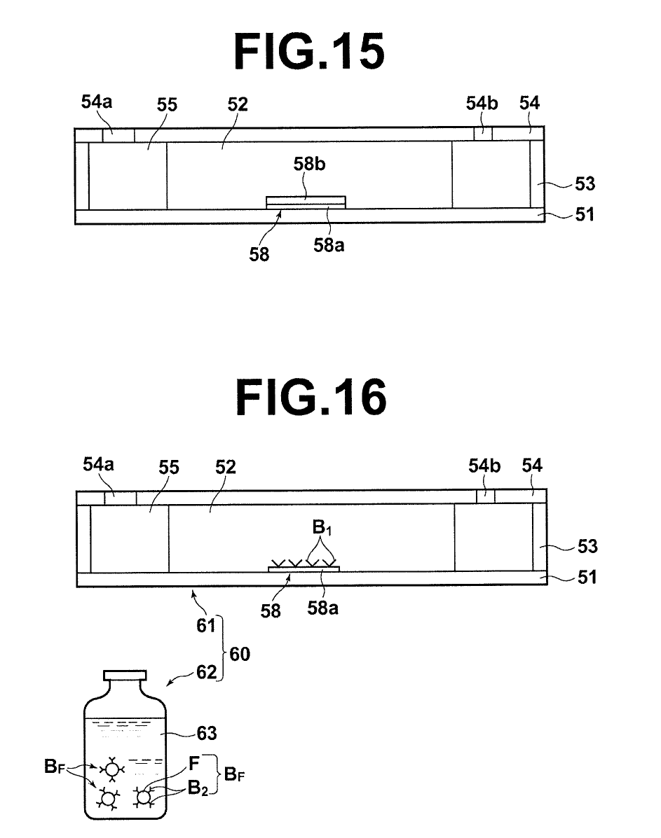

FIG. 15 is a diagram illustrating a sectional view of a sample cell used in the detection method and apparatus using optical field enhancement by an optical waveguide mode. The structure of the sample cell illustrated in FIG. 15 is substantially the same as the structure of the sample cell of the first embodiment, illustrated in FIGS. 11A and 11B. However, in FIG. 15, an optical waveguide layer 58b is further provided on the metal layer 58a in the sensor portion.

This sample cell may be used by appropriately immobilizing a first binding substance in the sensor portion and a fluorescent-label binding substance on the upstream side of the sensor portion.

### “Kit for Detection”

A kit for detection used in the detection method of the present invention will be described.

FIG. 16 is a schematic diagram illustrating the structure of a kit 60 for detecting fluorescence.

The kit 60 for detection includes a sample cell 61 and a solution 63 for labeling, which is injected into the flow path of the sample cell 61 together with the liquid sample or after the liquid sample flows down to perform fluorescence detection measurement. The solution 63 for labeling contains fluorescent-label binding substance B_{F} (hereinafter, referred to as “labeling secondary antibody B_{F}”) containing secondary antibody (second binding substance) B₂ that specifically binds to the antigen A and fluorescent substance F the surface of which has been modified with the secondary antibody B₂.

The sample cell 61 differs from the sample cell 50A of the second embodiment only in that a physical adsorption area, in which fluorescent-label binding substance B_{F} is physically adsorbed, is not provided in the sample cell 61. The remaining structure of the sample cell 61 is substantially the same as the structure of the sample cell 50A of the second embodiment.

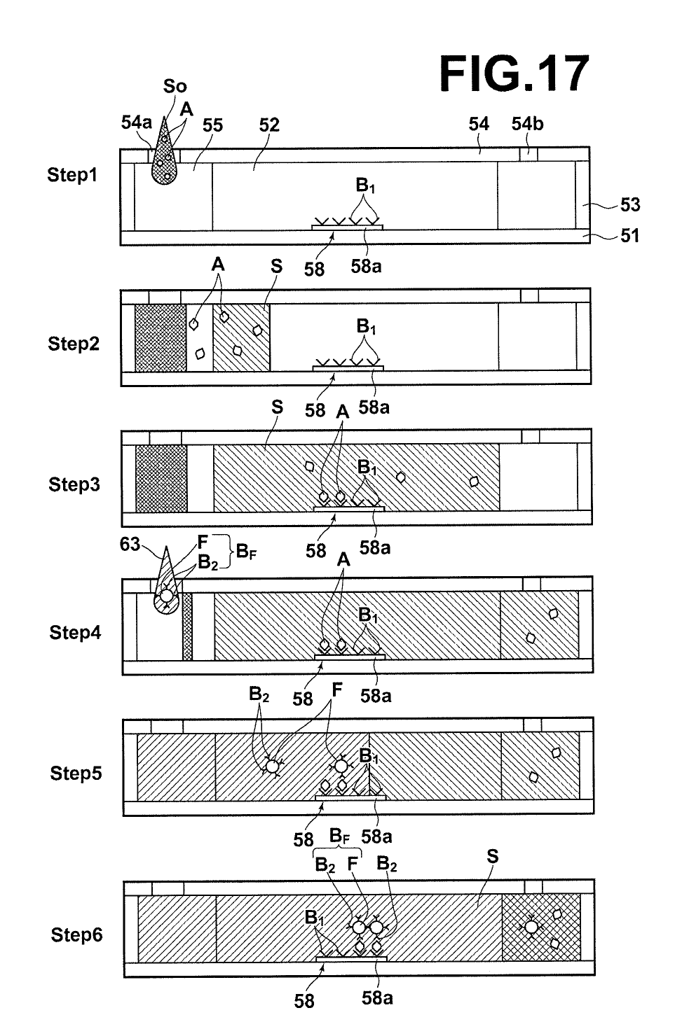

With reference to FIG. 17, assay procedures in the detection method of the present invention by using a sandwich method will be described. In the assay procedures, the kit 60 for detection of the present embodiment is used, and an assay is performed as to whether the blood (whole blood) contains an antigen, which is the detection target substance.

Step 1: Blood (whole blood) Sₒ, which is the assay target, is injected from an injection opening 54a. Here, a case in which the antigen that is the substance to be detected is contained in the blood Sₒ will be described. In FIG. 17, the whole blood Sₒ is indicated by a mesh.

Step 2: The whole blood Sₒ is filtered by the membrane filter 55, and large molecules, such as erythrocyte (red blood cells) and leukocyte (white blood cells), remain as a residue. Then, the blood (plasma, blood plasma) S after blood cells are removed by the membrane filter 55 penetrates into the flow path 52 by a capillary action. Alternatively, a pump may be connected to the air hole to accelerate reaction, thereby reducing detection time. The pump sucks the blood after blood cells are removed by the membrane filter 55 and pumps the sucked blood, thereby causing the blood to flow down in the flow path. In FIG. 17, the blood plasma S is indicated by a shadow.

Step 3: The blood plasma S gradually flows to the air hole 54b side along the flow path 52. The antigen A in the blood plasma S binds to the primary antibody B₁ that has been immobilized in the first measurement area 58.

Step 4: a solution 63 for labeling is injected from the injection opening 54a. The solution 63 for labeling contains labeling secondary antibody B_{F}.

Step 5: the labeling secondary antibody B_{F} penetrates into the flow path 52 by a capillary action. Alternatively, a pump may be connected to the air hole to accelerate reaction, thereby reducing detection time. The pump sucks the blood after blood cells are removed by the membrane filter 55 and pumps the sucked blood, thereby causing the blood to flow down in the path.

Step 6: The labeling secondary antibody B_{F} gradually flows down to the downstream side, and the secondary antibody B₂ of the labeling secondary antibody B_{F} binds to the antigen A. Consequently, a so-called sandwich in which the antigen A is sandwiched between the fragmented primary antibody B₁ and the secondary antibody B₂ is formed.

As described above, in steps 1 through 6, the blood is injected through the injection opening, and the antigen binds to the fragmented antibody and the secondary antibody. After steps 1 through 6, signals from the measurement area 58 are detected.

An example of a method for modifying the fluorescent substance with the secondary antibody and an example of a method for producing a solution for labeling will be described.

A solution containing MES buffer of 50 mM and an anti-hCG monoclonal antibody of 5.0 mg/mL (Anti-hCG 5008 SP-5, Medix Biochemica) is added to the fluorescent substance solution (diameter of the fluorescent substance is 500 nm, and the excitation wavelength is 502 nm, and the fluorescence wavelength is 510 nm) that has been prepared as described above, and stirred. Accordingly, the fluorescent substance is modified with the antibody.

Further, a WSC aqueous solution of 400 mg/mL (Product No. 01-62-0011, Wako Pure Chemical Industries, Ltd.) is added, and stirred at a room temperature.

Further, a Glycine aqueous solution of 2 mol/L is added, and stirred. Then, particles are precipitated by centrifugation.

Finally, the supernatant is removed, and PBS (pH 7.4) is added. An ultrasonic wash machine is used to redisperse the fluorescent substance the surface of which has been modified. Further, centrifugation is performed, and the supernatant is removed. Then, 500 μL of PBS (pH 7.4) solution of 1% BSA is added, and the fluorescent substance F the surface of which has been modified is redispersed to obtain a solution for labeling.

### (Design Modification Example of Kit for Examination)

As the sample cell that is used in the detection method and apparatus using the optical field enhancement by an optical waveguide mode, the sample cell as illustrated in FIG. 15 may be used. In the sample cell illustrated in FIG. 15, an optical waveguide layer 58b is further provided on the metal layer 58a of the sensor portion. Further, the fragmented primary antibody B₁ should be immobilized on the optical waveguide layer 58b of the sample cell.

Further, when an assay by a competition method is performed, instead of the fragmented primary antibody B₁ and the fragmented primary antibody B₂, the fragmented primary antibody (first binding substance) C₁ that specifically binds to the antigen A, which is the detection target substance, and the secondary antibody C₃ is immobilized on the sensor portion. Further, as the solution for labeling, a solution containing the fluorescent-label binding substance C_{F} should be used. The fluorescent-label binding substance C_{F} contains the secondary antibody (third binding substance) C₃ that does not bind to the antigen A, which is the detection target substance, but specifically binds to the fragmented primary antibody, and a fluorescent substance, the surface of which is modified with the secondary antibody C₃.

If the sample cell for detection and the kit for detection of the aforementioned embodiments are used, it is possible to easily carry out the detection methods of the present invention. Further, it is possible to effectively use the enhanced optical field, and to accurately detect presence and/or the amount of the detection target substance at high sensitivity.

### EXAMPLES

Examples of the present invention will be described.

### Example 1

The degree of dependence (dependence characteristic) of the electric field enhancement effect on a distance from the enhanced field generation surface (metal surface) was obtained by simulation for a detection apparatus structured in a manner similar to the detection apparatus illustrated in FIG. 1. In the simulation, it was assumed that in the detection apparatus, a PMMA prism having a gold film with a thickness of 50 nm on the surface thereof was used, and water was used as a solvent of the sample solution. Further, it was assumed that fluorescent beads having diameters of 310 nm were used as fluorescent label, and a laser beam having a wavelength of 656 nm entered at an incident angle of 72.5°. Further, a primary antibody immobilized on the sensor and a secondary antibody immobilized on the fluorescent label were assumed to be F(ab′)₂ for the primary antibody and F (ab′)₂ for the secondary antibody that have different epitopes from each other. Calculation was performed on the assumption that the primary antibody was directly immobilized on the sensor by an Au—S bond.

### Example 2

The dependence characteristic of the electric field enhancement effect on a distance from the enhanced field generation surface (metal surface) was simulated in a manner similar to Example 1 except that the F(ab′)₂ for the primary antibody was immobilized on the surface of the metal film through a SAM (thickness of the layer is 0.2 nm).

### Comparative Example 1

The dependence characteristic of the electric field enhancement effect on a distance from the enhanced field generation surface (metal surface) was simulated in a manner similar to Example 2 except that primary antibody (anti-hCG monoclonal antibody) before fragmentation was used as the primary antibody.

### Comparative Example 2

The dependence characteristic of the electric field enhancement effect on a distance from the enhanced field generation surface (metal surface) was simulated in a manner similar to Comparative Example 1 except that secondary antibody before fragmentation was used as the secondary antibody immobilized on the fluorescent label.

### (Result and Analysis)

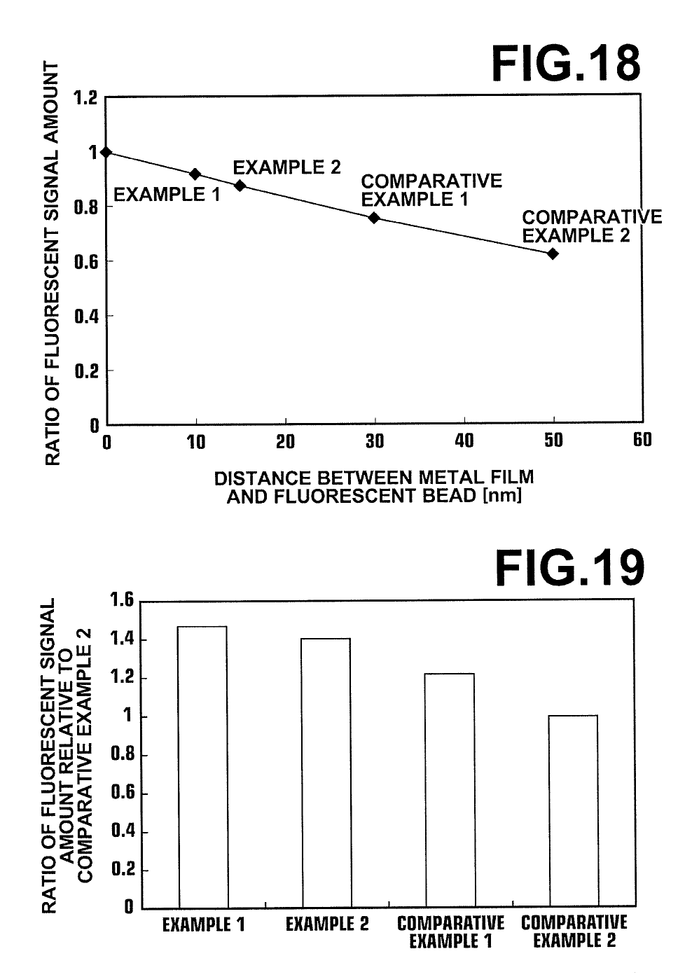

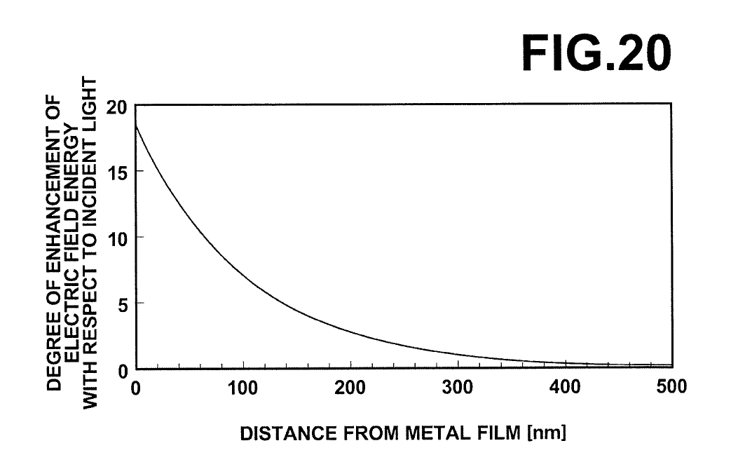

FIG. 18 illustrates the result of simulation. FIG. 18 shows the distance between the gold film and the lower end of the fluorescent substance (fluorescent bead) and the ratio of the strength of fluorescence at each distance relative to the strength of fluorescence obtained by directly placing the fluorescent bead on the surface of the gold film. The distance between the gold film and the lower end of the fluorescent substance (fluorescent bead) was estimated based on the primary antibody immobilized on the surface of the gold film, the secondary antibody immobilized on the fluorescent label, and the thickness of the SAM.

As FIG. 18 illustrates, when the distance from the gold film is approximately 10 nm, the strength of obtained fluorescence is approximately 90% of the fluorescent signal amount at the enhanced electric field surface. However, in comparative examples, which do not use fragmented antibodies as the primary antibodies, the strength of signals attenuates at least by 25%.

Further, FIG. 19 illustrates the relationship between the fluorescent signal amount obtained in Comparative Example 2, in which ordinary antibodies, which are not fragmented, are used for both of the primary antibody and the secondary antibody, and the fluorescent signal amounts obtained in Examples and Comparative Example 1.

In FIG. 19, the fluorescent signal amount of Comparative Example 1, in which a fragmented antibody is used only as the secondary antibody, is enhanced approximately by 20%, compared with Comparative Example 2, in which ordinary antibodies, which are not fragmented, are used for both of the primary antibody and the secondary antibody. This confirms that use of a fragmented antibody as the secondary antibody has an advantageous effect.

Further, in Examples 1 and 2, in which the fragmented antibodies are used for both of the primary antibody and the secondary antibody, the fluorescent signal amount is enhanced approximately by 40%, compared with Comparative Example 2. This confirms that use of a fragmented antibody as the primary antibody greatly enhances the signal amount.

Further, FIG. 19 shows that the fluorescent signal amount enhancement effect of Example 1, in which the fragmented primary antibody is directly immobilized without using a SAM, enhances the signal amount more efficiently than Example 2, in which the fragmented antibody is immobilized though a SAM. Binding the fragmented primary antibody directly without using the SAM is advantageous not only because the enhanced field is effectively used but also because the production process can be simplified by omitting the process of forming the SAM.

Accordingly, the advantageous effects of the present invention have been confirmed.

Further, the surface plasmon sensor of the present invention may be optionally used as a biosensor or the like.

What is claimed is:

1. A detection method comprising the steps of:
preparing a sensor chip including a dielectric plate and a sensor portion that has a metal layer deposited on a surface of the dielectric plate;
binding a labeling binding substance in an amount corresponding to the amount of a detection target substance contained in a liquid sample to the sensor portion by contacting the liquid sample with the sensor portion;
irradiating the sensor portion with excitation light to generate an enhanced optical field on the sensor portion; and
detecting the amount of the detection target substance based on the amount of light generated from a label of the labeling binding substance in the enhanced optical field, wherein a labeling substance including a light-responsive substance enclosed by a dielectric that transmits light output from the light-responsive substance is used as the label, and wherein the labeling binding substance binds to the sensor portion through a plurality of fragmented antibodies.

2. A detection method, as defined in claim 1, wherein the plurality of fragmented antibodies have been fragmented by a protease, and wherein a disulfide bond and/or a thiol group has been exposed by the fragmentation, and wherein the plurality of fragmented antibodies have antigenic determinant groups that can specifically bind to the detection target substance at least at one of the ends of each of the plurality of fragmented antibodies.

3. A detection method, as defined in claim 2, wherein at least one kind of antibody fragment selected from the group consisting of a Fab fragment, F(ab′)₂ fragment, and a Fab′ fragment is used as the plurality of fragmented antibodies.

4. A detection method, as defined in claim 1, wherein at least a part of the metal layer is exposed to a sample-contact surface of the sensor portion, and wherein the labeling binding substance binds to the sensor portion through the plurality of fragmented antibodies that have directly bounded to metal atoms in the at least part of the metal layer that is exposed.

5. A detection method, as defined in claim 1, wherein the labeling binding substance includes the labeling substance to the surface of which a fragmented antibody has bound, and wherein the fragmented antibody can specifically bind to the detection target substance and/or the plurality of fragmented antibodies.

6. A detection method as defined in claim 1, wherein plasmons are excited in the metal layer by irradiation with the excitation light to generate the optical field enhanced by the plasmons, and wherein the amount of the detection target substance is detected by using a fluorescent dye molecule as the light-responsive substance and by detecting, as the light generated from the label, fluorescence output by excitation of the fluorescent dye molecule.

7. A detection method as defined in claim 1, wherein plasmons are excited in the metal layer by irradiation with the excitation light to generate the optical field enhanced by the plasmons, and wherein the amount of the detection target substance is detected by using a fluorescent dye molecule as the light-responsive substance and by detecting, as the light generated from the label, radiation light that radiates from the other surface of the dielectric plate by newly inducing plasmons in the metal layer by fluorescence output by excitation of the fluorescent dye molecule.

8. A detection method, as defined in claim 1, wherein the sensor chip includes an optical waveguide layer deposited on the metal layer, and wherein an optical waveguide mode is excited in the optical waveguide layer by irradiation with the excitation light to generate the optical field enhanced by the optical waveguide mode, and wherein the amount of the detection target substance is detected by using a fluorescent dye molecule as the light-responsive substance and by detecting, as the light generated from the label, fluorescence output by excitation of the fluorescent dye molecule.

9. A detection method, as defined in claim 1, wherein the sensor chip includes an optical waveguide layer deposited on the metal layer, and wherein an optical waveguide mode is excited in the optical waveguide layer by irradiation with the excitation light to generate the optical field enhanced by the optical waveguide mode, and wherein the amount of the detection target substance is detected by using a fluorescent dye molecule as the light-responsive substance and by detecting, as the light generated from the label, radiation light that radiates from the other surface of the dielectric plate, the radiation light radiating by newly inducing plasmons in the metal layer by fluorescence output by excitation of the fluorescent dye molecule.

10. A detection method comprising the steps of:
preparing a sensor chip including a dielectric plate and a sensor portion that has a metal layer deposited on a surface of the dielectric plate and an optical waveguide layer deposited on the metal layer;
binding a labeling binding substance in an amount corresponding to the amount of a detection target substance contained in a liquid sample to the sensor portion by contacting the liquid sample with the sensor portion;
irradiating the sensor portion with excitation light to excite an optical waveguide mode in the optical waveguide layer to generate an enhanced optical field on the sensor portion by the optical waveguide mode; and
detecting the amount of the detection target substance based on the amount of light generated from a label of the labeling binding substance in the enhanced optical field, wherein the labeling binding substance binds to the sensor portion through a fragmented antibody.

11. A detection apparatus used in the detection method as defined in claim 1, the apparatus comprising:
a sensor chip including a dielectric plate and a sensor portion that has a metal layer deposited on a surface of the dielectric plate, the plurality of fragmented antibodies having bound onto the sensor portion;
an excitation-light irradiation optical system that irradiates the sensor portion with the excitation light; and
a light detection means that detects light generated from the label of the labeling binding substance in the enhanced optical field generated on the sensor portion by irradiation with the excitation light.

12. A detection apparatus used in the detection method as defined in claim 10, the apparatus comprising:
a sensor chip including a dielectric plate and a sensor portion that has a metal layer deposited on a surface of the dielectric plate, the plurality of fragmented antibodies having bound onto the sensor portion;
an excitation-light irradiation optical system that irradiates the sensor portion with the excitation light; and
a light detection means that detects light generated from the label of the labeling binding substance in the enhanced optical field generated on the sensor portion by irradiation with the excitation light.

13. A sample cell for detection used in the detection method as defined in claim 1, the sample cell comprising:
a base that has a flow path in which a liquid sample flows down;
an injection opening for injecting the liquid sample into the flow path, the injection opening being provided on the upstream side of the flow path;
an air hole for causing the liquid sample that has been injected from the injection opening to flow down toward the downstream side of the flow path, the air hole being provided on the downstream side of the flow path; and
a sensor chip portion provided between the injection opening and the air hole in the flow path, wherein the sensor chip portion includes a dielectric plate that is provided at least as a part of the inner wall of the flow path and a sensor portion that has at least a metal layer deposited on a sample-contact surface of the dielectric plate, wherein the plurality of fragmented antibodies have bound to a surface of the sensor portion opposite to the dielectric-plate-side surface of the sensor portion.

14. A sample cell for detection used in the detection method as defined in claim 10, the sample cell comprising:
a base that has a flow path in which a liquid sample flows down;
an injection opening for injecting the liquid sample into the flow path, the injection opening being provided on the upstream side of the flow path;
an air hole for causing the liquid sample that has been injected from the injection opening to flow down toward the downstream side of the flow path, the air hole being provided on the downstream side of the flow path; and
a sensor chip portion provided between the injection opening and the air hole in the flow path, wherein the sensor chip portion includes a dielectric plate that is provided at least as a part of the inner wall of the flow path and a sensor portion that has at least a metal layer deposited on a sample-contact surface of the dielectric plate, wherein the plurality of fragmented antibodies have bound to a surface of the sensor portion opposite to the dielectric-plate-side surface of the sensor portion.

15. A sample cell for detection, as defined in claim 13, wherein an optical waveguide layer is provided on the metal layer in the sensor portion.

16. A sample cell for detection, as defined in claim 13, wherein the labeling binding substance is immobilized in the flow path on the upstream side of the sensor portion, and wherein the labeling binding substance includes, as the label, a labeling substance that contains a light-responsive substance enclosed by a dielectric that transmits light output from the light-responsive substance.

17. A sample cell for detection, as defined in claim 14, wherein the labeling binding substance is immobilized in the flow path on the upstream side of the sensor portion, and wherein the labeling binding substance includes, as the label, a labeling substance that contains a light-responsive substance enclosed by a dielectric that transmits light output from the light-responsive substance.

18. A kit for detection comprising:
a sample cell as defined in claim 13; and
a solution for labeling, wherein the solution for labeling contains the labeling binding substance that includes, as the label, a labeling substance that includes a light-responsive substance enclosed by a material that transmits light output from the light-responsive substance, and wherein the solution for labeling is injected into the flow path to flow down the flow path together with the liquid sample or after the liquid sample has flowed down the flow path.

19. A kit for detection comprising:
a sample cell as defined in claim 14; and
a solution for labeling, wherein the solution for labeling contains the labeling binding substance that includes, as the label, a labeling substance that includes a light-responsive substance enclosed by a material that transmits light output from the light-responsive substance, and wherein the solution for labeling is injected into the flow path to flow down the flow path together with the liquid sample or after the liquid sample has flowed down the flow path.
